(19) United States
(12) Patent Application Publication
(10) Pub. No.: US 2025/0366723 A1
(43) Pub. Date: Dec. 4, 2025

(54) SYSTEM FOR AND METHOD OF MEASURING BLOOD PRESSURE NON-INVASIVELY WITH LIGHT

(71) Applicant: The General Hospital Corporation, Boston, MA (US)

(72) Inventors: Maria A. Franceschini, Charlestown, MA (US); Marco Renna, Charlestown, MA (US); Mitchell Robinson, Charlestown, MA (US); Kuan Wu, Charlestown, MA (US)

(21) Appl. No.: 18/875,000

(22) PCT Filed: Jun. 15, 2023
(86) PCT No.: PCT/US2023/068553

§ 371 Date: Dec. 13, 2024

Related U.S. Application Data
(60) Provisional application No. 63/352,382, filed on Jun. 15, 2022.

(51) Int. Cl. A61B 5/021; A61B 5/00; A61B 5/024

(52) U.S. Cl. CPC A61B 5/021; A61B 5/02416; A61B 5/7264; A61B 2560/0223

(57) ABSTRACT

Optical patient monitoring systems are disclosed. The system may comprise an optical coupling system configured to transmit to and receive light signals from one or more locations on a subject; an optical processing system configured to generate optical data using the received light signals; and a computer programmed to receive the optical data; determine, using the optical data, at least one indicator of blood pressure; estimate an estimated blood pressure using the at least one indicator of blood pressure; and generate a report indicative of the estimated blood pressure. The at least one indicator of blood pressure comprises one or more of near-infrared spectroscopy (NIRS) data; photoplethysmography (PPG) data, diffuse correlation spectroscopy (DCS) data, speckle contrast optical spectroscopy (SCOS) data, speckleplethysmography (SPG) data, first derivative PPG data, second derivative PPG data, first derivative SPG data, second derivative SPG data, inflow (Fin) data, outflow (Fout) data, heart rate data, physiological data, and combinations thereof. Methods for estimating blood pressure are also disclosed.

### CROSS-REFERENCE TO RELATED APPLICATIONS

The present application claims priority to U.S. Provisional Patent Application No. 63/352,382, filed Jun. 15, 2022, the entire contents of which are hereby incorporated by reference.

### STATEMENT REGARDING FEDERALLY SPONSORED RESEARCH

This invention was made with government support under 5R21EB028626-02 and 1R21AG072481-01 awarded by National Institutes of Health. The government has certain rights in the invention.

### BACKGROUND OF THE INVENTION

Each heartbeat sends a pressure wave through the vasculature, which causes local changes in both blood volume and blood flow. Near-infrared spectroscopy (NIRS), diffuse correlation spectroscopy (DCS), and speckle contrast optical spectroscopy (SCOS) are non-invasive, diffuse optical techniques that can measure these changes in blood volume and blood flow. Each method involves sending light into the tissue, measuring light that scatters back to the surface of the skin, and inferring hemodynamic changes based on the changes in the optical signals.

The changes in blood volume modulate the measured light intensity (NIRS), and the pulsatile component of the optical signal is termed photoplethysmography (PPG). PPG is used in pulse oximetry and health tracker devices to measure arterial blood oxygenation, heart rate, and heart rate variability.

Using coherent illumination (e.g. laser illumination), changes in blood flow can be measured by the quantification of the changes in the detected speckle pattern. By monitoring either changes in the temporal autocorrelation of the detected intensity at the microsecond to millisecond timescale (DCS) or the spatial blurring of the detected speckle pattern (SCOS), both methods relay information about the motion of cells in the vasculature, and thus the blood flow can be measured. The pulsatile component of the blood flow signal is termed speckleplethysmography (SPG).

Both the PPG and SPG waveforms and the time derivatives of the PPG and SPG waveforms have features that can be important to the determination of blood pressure. The connection between

$$\frac{d P P G}{dt}$$

and blood flow is detailed in WO2016/164894 the entire contents of which is expressly incorporated herein by reference.

However, important features including the systolic peak (P₁) and the pre-dicrotic peak (P₂)—also known as the tidal or post-systolic wave—NIRS data, DCS data, and SCOS data have failed to be previously considered. As such, there is an unmet need in the art to consider these elements in estimating, measuring, and monitoring blood pressure and other biometrics.

### BRIEF SUMMARY OF THE INVENTION

Near-infrared spectroscopy (NIRS), diffuse correlation spectroscopy (DCS), and speckle contrast optical spectroscopy (SCOS) are non-invasive, diffuse optical techniques that can measure changes in blood volume and blood flow. Changes in blood volume (during a cardiac cycle, for example) modulate the measured light intensity (NIRS), and the pulsatile component of the optical signal is termed photoplethysmography (PPG). Changes in blood flow can be measured by the quantification of the changes in the detected speckle pattern. The pulsatile component of the blood flow signal is termed speckleplethysmography (SPG). Both the PPG and SPG waveforms have features that can be important to the determination of blood pressure. Further, the systolic peak (P1) and the pre-dicrotic peak (P2)—also known as the tidal or post-systolic wave—NIRS data, DCS data, and SCOS data have failed to be considered important in the prior art. The present disclosure at least meets this need in the art. Further, the present disclosure describes at least the following innovations over the prior art: the use of speckle imaging features (e.g. the P2/P1 feature) and the need for a placement of the probe on the forehead, neck, and/or earlobe, the use of the PPG derivative signal and the specific features derived from those signals, the use of beat shape as opposed to pulse transit time, and the use of time domain features over frequency domain features in the derivation of pressure.

In some aspects, the present disclosure provides optical patient monitoring systems comprising an optical coupling system configured to transmit and receive light signals at one or more locations on a subject; an optical processing system configured to generate optical data using the received light signals; and a computer programmed to receive the optical data; determine, using the optical data, at least one indicator of blood pressure; estimate an estimated blood pressure using the at least one indicator of blood pressure; and generate a report indicative of the estimated blood pressure. In some aspects, the present disclosure provides methods for estimating blood pressure, the method comprising transmitting and receiving light signals from one or more locations on a subject using an optical coupling system configured to transmit and receive light signals; determining, using optical data generated using the received light signals using an optical processing system, at least one indicator of blood pressure; estimating blood pressure of the subject using the at least one indicator of blood pressure.

In some aspects, the at least one indicator of blood pressure comprises one or more of: near-infrared spectroscopy (NIRS) data; photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative

$$\frac{{d}^{2} P P G}{{dt}^{2}}$$

data, third derivative

$$\frac{{d}^{3} P P G}{{dt}^{3}}$$

data, first derivative

$$\frac{d S P G}{dt}$$

data, second derivative

$$\frac{{d}^{2} S P G}{{dt}^{2}}$$

data, inflow (Fᵢₙ) data, outflow (Fₒᵤₜ) data, heart rate data, physiological data, and combinations thereof, and in some aspects the at least one indicator of blood pressure comprises one or more of: photoplethysmography (PPG) data, first derivative

$$\frac{d P P G}{dt}$$

data, second derivative

$$\frac{{d}^{2} P P G}{{dt}^{2}}$$

data, and third derivative

$$\frac{{d}^{3} P P G}{{dt}^{3}}$$

data, and the optical coupling system comprises a light source comprising at least one of an LED or photodiode in a NIRS device, pulse oximeter or cerebral oximeter. In some aspects, the physiological data comprises at least one of electrocardiogram (ECG) data, electroencephalogram (EEG) data, near infrared spectroscopy (NIRS) data, measured blood pressure data, respiratory data, hemoglobin data, pulse oximetry data, tissue oxygenation data, heart rate data, or combinations thereof. In some aspects, the at least one indicator of blood pressure comprises a ratio of the magnitude of a pre-dicrotic peak (P₂) and a systolic peak (P₁) for at least one individual cardiac cycle in at least one of a PPG curve, a

$$\frac{dPPG}{dt}$$

curve, an SPG curve, or a

$$\frac{dSPG}{dt}$$

curve. In some aspects, the at least one indicator of blood pressure comprises one or more of subject height, subject weight, subject age, subject gender, subject body position data, subject location measured data, and combinations thereof. In some aspects, the optical data is acquired over a plurality of cardiac cycles, and in some aspects, the optical data is acquired at a temporal resolution greater than a pulsatile frequency of the subject. The blood pressure may be at least one of systolic blood pressure (SBP), diastolic blood pressure (DBP), and mean arterial pressure (MAP).

The computer of the system may be configured to estimate the estimated blood pressure by performing a calibration against a measured blood pressure data set, wherein the measured blood pressure data set may be acquired by at least one of continuous measurements, non-continuous measurements, invasive (intra-arterial) blood pressure monitoring, non-invasive volume-clamp method, cuff point measurements, automated oscillometry, manual auscultation, and combinations thereof, and the computer may be configured to apply a machine learning algorithm to estimate the estimated blood pressure by analyzing a measured blood pressure data set against at least one of: NIRS data, PPG data, DCS data, SCOS data, SPG data, first derivative

$$\frac{dPPG}{dt}$$

data, second derivative

$$\frac{{d}^{2} PPG}{{dt}^{2}}$$

data, first derivative

$$\frac{dSPG}{dt}$$

data, second derivative

$$\frac{{d}^{2} SPG}{{dt}^{2}}$$

data, inflow (Fᵢₙ) data, outflow (Fₒᵤₜ) data, heart rate data, a ratio of the magnitude of a pre-dicrotic peak (P₂) and a systolic peak (P₁), and combinations thereof, wherein the machine learning algorithm approach may be at least one of random forests, support vector machines, gaussian process regression, and deep belief networks, and combination thereof. In some aspects, the one or more locations on a subject comprise the subject's head, forehead, neck, earlobe, and combinations thereof. In some aspects, the heart rate data comprises instantaneous heart rate data. In some aspects, the at least one indicator of blood pressure is acquired during a portion of each cardiac cycle. In some aspects, the computer is configured to or the method further comprises determining an effectiveness of an administered treatment using the estimated blood pressure and/or determining a condition of the subject based on the estimated blood pressure.

In some aspects, the transmitted light signal is from a light-emitting diode (LED) source, laser, or combinations thereof, the light signals are transmitted from an optical source and received by a photodetector that are placed between 1 mm and 40 mm apart, and/or the light signals have wavelengths between about 300 nm and about 2000 nm. In some aspects, the optical source is coherent, and in some aspects, the level of coherence and source output power is determined by the separation of the source and detector. In some aspects, the light signal is detected by a detector selected from the group comprising PIN photodiodes, avalanche photodiodes (APDs), single-photon avalanche diodes (SPADs), photomultiplier tubes (PMTs), silicon photomultipliers (SiPMs), charge-coupled device (CCD), complementary metal-oxide-semiconductor (CMOS) camera sensors, and combinations thereof, while in some aspects the light signal is detected by a detector selected from the group comprising single-photon avalanche diodes (SPADs), avalanche photodiodes (APDs), silicon photomultipliers (SiPMs), super conducting nanowire detectors (SNSPDs), photomultiplier tubes (PMTs), high frame rate photodiodes, camera sensors, and combinations thereof, while in some aspects the light signal is detected by a detector selected from the group comprising photodiodes arrays, electron multiplying cameras, intensified cameras, standard CCD cameras, CMOS cameras, and combinations thereof.

In some aspects, the method further comprises generating a report indicative of the estimated blood pressure.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates a comparison of the PPG, SPG, and

$$\frac{dPPG}{dt}$$

waveforms for a single cardiac cycle.

FIG. 2 illustrates a comparison of the shapes of the PPG curves (left figures) and the

$$\frac{dPPG}{dt}$$

curves (right figures) for the forehead (upper figures) and fingertip (lower figures). In the fingertip curves, P2 resides very close to 0, and as a result, the ratio of P2/P1 is not a reliable measure in the fingertip, though consistent changes in the P₂ feature are seen in the forehead.

FIG. 3 illustrates a comparison of the shape of the SPG curve at different mean arterial pressures. The ratio of P2/P1 shows a monotonic relationship with blood pressure that allows for estimation given a calibration.

FIG. 4 illustrates a demonstration of the multivariate regression method on the recovery of blood pressure across three different subjects across several tasks, including breath holding (BH), hyperventilation (HV), cold pressor (CPT), and biking. In the first subject, the distribution of heart rate was not varied enough to affect the fitting, and the linear regression including only the P2/P1 feature fit the data nearly as well as the multivariate model. In the other two subjects, the biking (subject 2) and hyperventilation (subject 3) conditions introduced a change in heart rate but not the same change in beat morphology for changes in blood pressure. The improvement in the fit of the data demonstrates the value of including the heart rate in the blood pressure estimation process.

FIG. 5 illustrates example results of the estimated mean arterial pressure (MAP) from the gaussian process regression model using SPG data from a single subject. Inputs to the model were SPG signals during individual cardiac cycles and the heart rate during each cycle. For individual subjects with calibration, this method yields very high accuracy estimates of blood pressure (−0.0474±1.583 mmHg).

FIG. 6 illustrates an example measurement setup (note that the two probes are not co-localized). Left (601-609): SNSPD-based DCS for longer separations (2.0 and 3.0 cm) and Si SPAD-based DCS for the short separation (0.5 cm). A 1064 nm CW-laser (601) transmits a light signal through a 50-50 splitter (602) which branches into two 100 mW signals that transmit through an 5, 20, and 30 mm SD separation DCS probe (603) comprising detectors (604) and a dual illumination source (605). One signal set then transmits through a 5 mm Si SPAD (606) while the other transmits through four (one 25 mm, three 30 mm) SNSPDs (607). The signals transmit to a correlator (608) connected to a computer (609) by USB. Right (610-615): FlexNIRS for hemoglobin concentration and SO2. A piece of white electrical tape was applied to the short SDsep PD to attenuate the light. An Android tablet (610) transmits signal via Bluetooth to a BLE module (611) and AFE module (612) to an 8, 28, and 33 mm SD separation FlexNIRS probe (613) comprising PIN photodiode detectors (614) and dual-color (735 and 850 nm) LEDs (615). The signal is transmitted back through the AFE module (612) and BLE module (611) back to the Android tablet (610). On Subject (616-619): biometrics are measured by Finapres (616), ECG (617), respiration belt (618), and pulse oximeter (619).

FIG. 7 illustrates average pulsatile waveforms of a representative subject (#5) during the measured conditions (A). The features for PWA are listed on the baseline panel. (B) pCBFi (used interchangeably with SPG) and pCBFi-fit comparison are shown, and inflow and outflow contribution to the pulsatile flow on the same subject.

FIG. 8 illustrates fit quality R2 (A) and fitting parameters C2/C1 (B) for the three tasks. Different color represents different subjects. The asterisks indicate paired-sample sign test significant difference levels. (*: p<0.05, **: p<0.01, ***: p<0.001).

FIG. 9 illustrates fitting parameters C1, C2 and C3 across subjects and tasks (A-C). The asterisks indicate paired-sample sign test significant difference levels. (*: p<0.05, **: p<0.01, paired-sample sign test).

FIG. 10 illustrates PWA peaks extracted from pCBFi-fit. The amplitude of the peaks was calculated relative to the diastolic blood flow. Each color represents a subject as indicated in the legend. The asterisks indicate paired-sample sign test significant difference levels. (A) Systolic peak P1 and (B) the secondary systolic peak P2, and (C) the ratios between the two peaks across subjects and tasks. (*: p<0.05, **: p<0.01, paired-sample sign test).

FIG. 11 illustrates SNSPD-DCS pCBFi and NIRS-PPG pCBFi-fit average and standard deviation of pulsatile signals during 60-s baseline (A). (B) Corresponding coefficient of variation for the 10 subjects (violin plot).

FIG. 12 illustrates a blood pressure measurement setup for a subject (1201) comprising a laser (1202) that transmits a light signal through a DCS-SPG probe (1203). Signals may then transmit divergently through Si SPAD (1204), for example, and SNSPDs (1205), for example. The signals may transmit to a correlator (1206) connected to a computer (1207) by USB, for example. An Android tablet (1208), for example, transmits signal via Bluetooth to a BLE module (1209), for example, and AFE module (1210), for example, NIRS-PPG probe (1211). The signal is transmitted back through the AFE module (1210) and BLE module (1209) back to the Android tablet (1208). Estimation of blood pressure may be conducted through continuous or non-continuous non-invasive volume-clamp methods including Finapres devices, for example (1212), and optional respiration belt (1213), pulse oximeter (1214), and/or ECG (1215) may be used. Sensors may be placed at exemplary locations such as forehead (1216), earlobe (1217), and/or neck (1218).

FIG. 13 illustrates a simplified block diagram of the hardware architectures of a combined NIRS/SCOS wearable device to measure ABP in the forehead: (A) Fiber-based SCOS; (B) Second generation FlexNIRS with optical inserts and sync for SCOS; (C) Integrated contact-SCOS-FlexNIRS with a low-cost monochromatic CMOS detector mounted directly in the FlexNIRS probe. BS: beam splitter, PD: photodiode, SS: SCOS at short separation, red: SCOS fibers, gray: SS SCOS, orange: FlexNIRS components. (D) PCB layout render of our current FlexNIRS. To add SCOS in the center of the probe the lines will be rerouted to the sides.

FIG. 14 illustrates a scatterplot of estimated vs actual mean blood pressure measured in the ear of a subject using an ear clip NIRS device. Mean arterial pressure was estimated using a

$$\frac{dPPG}{dt}$$

multivariate regression method with P₂/P₁ and heart rate as inputs.

FIG. 15 illustrates the continuous measurement of blood pressure (mean, systolic, and diastolic) with an a-line in a patient during a carotid enterectomy surgery. The

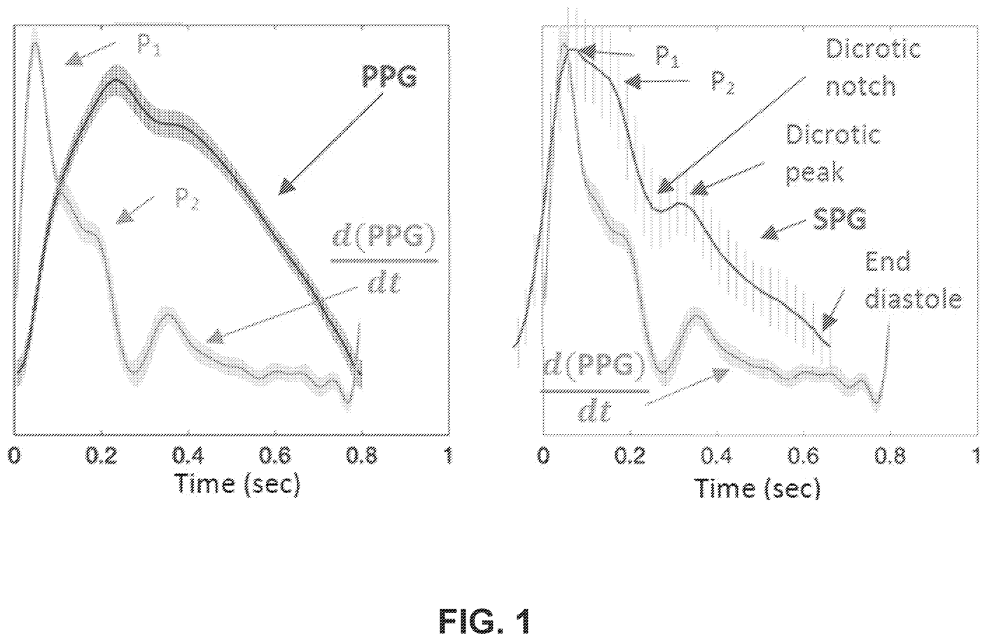

$$\frac{dPPG}{dt}$$

P₂/P₁ ratio measured with the FlexNIRS in the forehead of the patient (ipsilateral to the surgery side) estimated a systolic blood pressure that correlated to the a-line-measured blood pressure.

### DETAILED DESCRIPTION OF THE INVENTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Further, as used herein, ranges that are between two particular values should be understood to expressly include those two particular values. For example, “between 0 and 1” means “from 0 to 1” and expressly includes 0 and 1 and anything falling inside these values. Also, as used herein “about” means±20% of the stated value, and includes more specifically values of ±10%, ±5%, ±2%, ±1%, and ±0.5% of the stated value.

Each heartbeat sends a pressure wave through the vasculature, which causes local changes in both blood volume and blood flow. Near-infrared spectroscopy (NIRS), diffuse correlation spectroscopy (DCS), and speckle contrast optical spectroscopy (SCOS) are non-invasive, diffuse optical techniques that can measure these changes in blood volume and blood flow. Each method involves sending light into the tissue, measuring light that scatters back to the surface of the skin, and inferring hemodynamic changes based on the changes in the optical signals.

The changes in blood volume (during a cardiac cycle, for example) modulate the measured light intensity (NIRS), and the pulsatile component of the optical signal is termed photoplethysmography (PPG). PPG is used in pulse oximetry and health tracker devices to measure arterial blood oxygenation, heart rate, and heart rate variability. PPG can also be used to measure blood pressure, not to be limited to the exemplary NIRS-PPG embodied throughout the disclosure, particularly in Example 2.

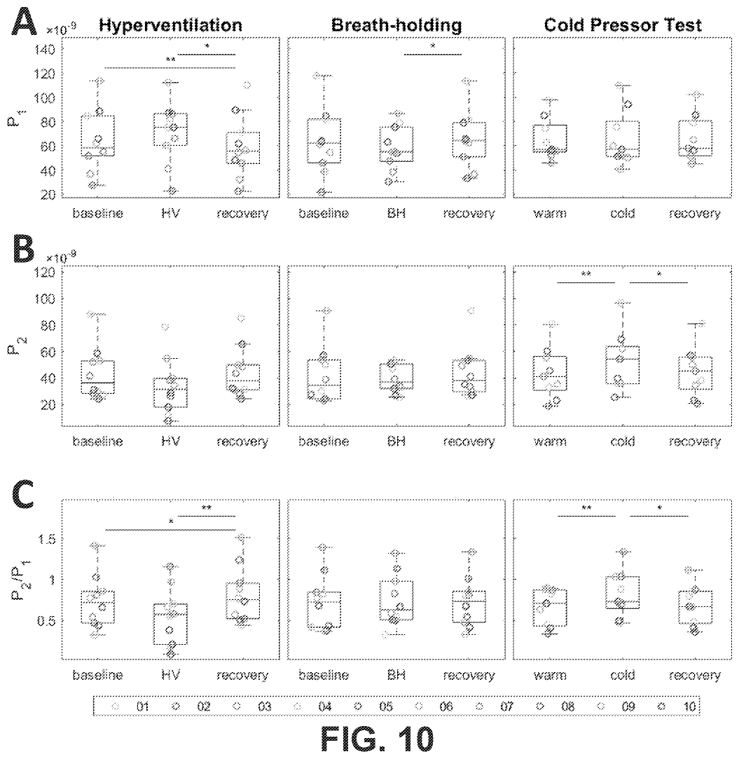

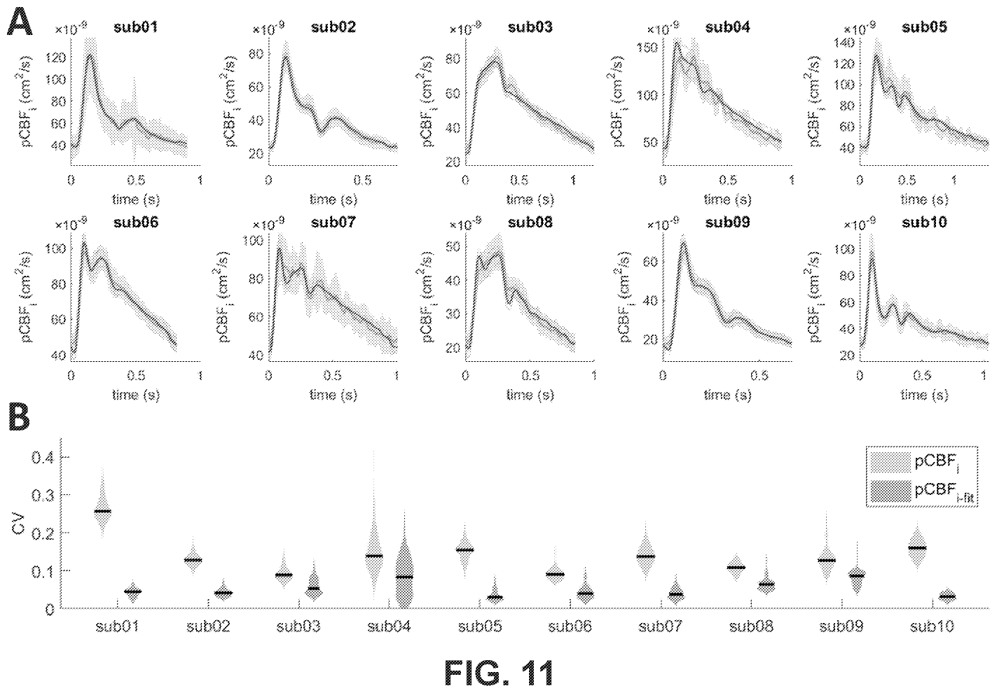

Using coherent illumination (e.g. laser illumination), changes in blood flow can be measured by the quantification of the changes in the detected speckle pattern. By monitoring either changes in the temporal autocorrelation of the detected intensity at the microsecond to millisecond timescale (DCS) or the spatial blurring of the detected speckle pattern (SCOS), both methods relay information about the motion of cells in the vasculature, and thus the blood flow can be measured. The pulsatile component of the blood flow signal is termed speckleplethysmography (SPG). One non-limiting example of SPG may be pulsatile cerebral blood flow index (pCBFi), which may embody a more specific recital of SPG throughout this disclosure, particularly in Example 2 and FIG. 11.

Both the PPG and SPG waveforms have features that can be important to the determination of blood pressure. The connection between

$$\frac{dPPG}{dt}$$

and blood flow is detailed in WO2016/164894, the entire disclosure of which is expressly incorporated herein by reference. Further, the systolic peak (P₁) and the pre-dicrotic peak (P₂)—also known as the tidal or post-systolic wave—NIRS data, DCS data, and SCOS data have failed to be considered important in the prior art. The present disclosure at least meets this need in the art.

In one aspect, the present disclosure provides optical patient monitoring systems comprising an optical coupling system configured to transmit to and receive light signals from one or more locations on a subject; an optical processing system configured to generate optical data using the received light signals; and a computer programmed to receive the optical data; determine, using the optical data, at least one indicator of blood pressure; estimate an estimated blood pressure using the at least one indicator of blood pressure; and generate a report indicative of the estimated blood pressure. The at least one indicator of blood pressure may comprise one or more of: near-infrared spectroscopy (NIRS) data; photoplethysmography (PPG) data, diffuse correlation spectroscopy (DCS) data, speckle contrast optical spectroscopy (SCOS) data, speckleplethysmography (SPG) data, first derivative

$$\frac{dPPG}{dt}$$

data, second derivative

$$\frac{{d}^{2} PPG}{{dt}^{2}}$$

data, third derivative

$$\frac{{d}^{3} PPG}{{dt}^{3}}$$

data, first derivative

$$\frac{dSPG}{dt}$$

data, second derivative

$$\frac{{d}^{2} SPG}{{dt}^{2}}$$

data, inflow (Fᵢₙ) data, outflow (Fₒᵤₜ) data, heart rate data, a ratio of the magnitude of a pre-dicrotic peak (P₂) and a systolic peak (P₁), and combinations thereof. Further, the physiological data may comprise at least one of electrocardiogram (ECG) data, electroencephalogram (EEG) data, near infrared spectroscopy (NIRS) data, measured blood pressure data, respiratory data, hemoglobin data, pulse oximetry data, tissue oxygenation data, heart rate data, or a combination thereof. In some cases, the at least one indicator of blood pressure comprises at least PPG data and SPG data. In some cases, the at least one indicator of blood pressure comprises at least DCS data, NIRS data, and SCOS data. In some cases, the at least one indicator of blood pressure may comprise or further comprise a ratio of the magnitude of a pre-dicrotic peak (P2) and a systolic peak (P1) for at least one individual cardiac cycle in at least one of a PPG curve, a

$$\frac{dPPG}{dt}$$

curve, an SPG curve, or a

$$\frac{dPSG}{dt}$$

curve. The ratio of the magnitude of P2 and P1 for individual cardiac cycles in either the

$$\frac{dPPG}{dt}$$

or SPG curves can be calculated. The instantaneous heart rate during each cardiac cycle can be estimated as the inverse of the duration of the cardiac cycle. Estimations of blood pressure can then be found by performing multivariate linear regression, for example by fitting a linear model of the form

$$BP = {c}_{1} + {c}_{2} * \frac{{P}_{2}}{{P}_{1}} + {c}_{3} * HR .$$

In other instances, the normalized optical waveforms are passed through machine learning approaches to generate estimates of blood pressure. In some cases, the at least one indicator of blood pressure may comprise or further comprise one or more of subject height, subject weight, subject age, subject gender, subject body position data, subject location measured data and/or at a temporal resolution greater than a pulsatile frequency of the subject. In some cases, the pulsatile frequency is the pulsatile frequency of cerebral blood flow. The at least one indicator of blood pressure may be acquired during a portion of each cardiac cycle.

“Cardiac cycle” may refer to the performance of the heart from the beginning of one heartbeat to the beginning of the next and can consist of two periods: one during which the heart muscle relaxes and refills with blood, which may be called diastole, following a period of robust contraction and pumping of blood, which may be called systole. “Curves” referred to herein may refer to the shape, or “beat shape”, of the optical data that is processed and illustrated through graphical means. “Pulsatile frequency” may refer to the regularity or period of pulses of heart rate activity. “Temporal resolution” may refer to the period duration or scale on which measurements are made. Therefore, “a temporal resolution greater than a pulsatile frequency of cerebral blood flow” may refer to measurements made at smaller increments than the increments between cerebral pulses. “Computer” may refer to a variety of devices capable of storing and/or processing data, which may be in binary form, according to instructions given to it in a variable programs and is understood by those of skill in the art to include devices not limited to desktop and laptop computers, tablets, and phones.

The optical coupling system may comprise an optical coupler which may comprise a semiconductor device, which is designed to transfer electrical signals by using light waves in order to provide coupling with electrical isolation between circuits or systems. Optical data may refer to data collected through at least the means disclosed herein. The means for collecting these data are commonly known in the art, some of which are described in the Examples.

Estimated blood pressure may be at least one of systolic blood pressure (SBP), diastolic blood pressure (DBP), and mean arterial pressure (MAP).

The computer may be configured to estimate the estimated blood pressure by performing a calibration against a measured blood pressure data set, and the measured blood pressure data set may be acquired by at least one of continuous measurements, invasive (intra-arterial) blood pressure monitoring, non-invasive volume-clamp method (Finapres device, for example), cuff point measurements, automated oscillometry, manual auscultation, and combinations thereof. The computer may be configured to apply a machine learning algorithm to estimate the estimated blood pressure by analyzing a measured blood pressure data set against at least one of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative

$$\frac{dPPG}{dt}$$

data, second derivative

$$\frac{{d}^{2} PPG}{{dt}^{2}}$$

data, third derivative

$$\frac{{d}^{3} PPG}{{dt}^{3}}$$

data, first derivative

$$\frac{dSPG}{dt}$$

data, second derivative

$$\frac{{d}^{2} SPG}{{dt}^{2}}$$

data, inflow (Fᵢₙ) data, outflow (Fₒᵤₜ) data, heart rate data, a ratio of the magnitude of a pre-dicrotic peak (P₂) and a systolic peak (P₁), and combinations thereof.

The machine learning model may include a trained machine learning model (e.g., a trained neural network) that analyzes and classifies data received and/or processed in the disclosed systems. For example, the machine learning controller or model may be configured to apply a machine learning algorithm to estimate the estimated blood pressure by analyzing a measured blood pressure data set against at least one of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative

$$\frac{dPPG}{dt}$$

data, second derivative

$$\frac{{d}^{2} PPG}{{dt}^{2}}$$

data, third derivative

$$\frac{{d}^{3} PPG}{{dt}^{3}}$$

data, first derivative

$$\frac{dSPG}{dt}$$

data, second derivative

$$\frac{{d}^{2} SPG}{{dt}^{2}}$$

data, inflow (Fᵢₙ) data, outflow (Fₒᵤₜ) data, heart rate data, a ratio of the magnitude of a pre-dicrotic peak (P₂) and a systolic peak (P₁), and combinations thereof. In this non-limiting example, measured optical data is measured in addition to physical measurement of actual blood pressure (including means recited herein), values of which are used to train a machine to identify features of relevance in the waveforms found in optical data that serve as indicators of blood pressure. Machine learning may be used to determine an algorithm to establish a correlation between physically measured blood pressure and blood pressure estimated from optically obtained indicators of blood pressure as disclosed herein.

The machine learning algorithm approach may be at least one of random forests, support vector machines, gaussian process regression, and deep belief networks, and combination thereof. Data collected in the systems disclosed herein may be stored on a server and/or maintained on a database. The data may be used to train a machine learning model which may be used to develop the machine learning algorithm. Machine learning may refer to the general use of artificial intelligence (AI).

The machine learning algorithm may be developed through trained machine learning controls (e.g., trained machine learning model and/or algorithms), artificial intelligence controls (e.g., rules and/or other control logic implemented in an artificial intelligence model and/or algorithm), and the like. In some embodiments, the receives data for constructing, training, adjusting, or executing a machine learning model (e.g., a machine learning controller). That is, the machine learning model or controller may be software or a set of instructions executed by a server processor or other computer. In some cases, the machine learning controller or model includes a separate processor and memory to execute the software or instructions to implement the functionality of the machine learning controller or model.

The machine learning controller implements a machine learning program, algorithm, or model. In some implementations, the machine learning controller is configured to construct a model (e.g., building one or more algorithms) based on example inputs, which may be done using supervised learning, unsupervised learning, reinforcement learning, ensemble learning, active learning, transfer learning, or other suitable learning techniques for machine learning programs, algorithms, or models. Additionally or alternatively, the machine learning controller may be configured to modify a machine learning program, algorithm, or model; to active and/or deactivate a machine learning program, algorithm, or model; to switch between different machine learning programs, algorithms, or models; and/or to change output thresholds for a machine learning program, algorithms, or model.

The machine learning controller or model may be a static machine learning controller or model, a self-updating machine learning controller or model, an adjustable machine learning controller or model, or the like.

The computer may include any suitable computer device, including a standalone computer such as a laptop or desktop, a handheld device such as a pad or tablet, as well as smaller devices such as phones, watches, etc.

Calibration may be performed by means known in the art, some of which are described in the Examples and may rely on a calibration period where both optical measurements and blood pressure measurements are taken concurrently.

The one or more locations on a subject may comprise the subject's forehead, neck or earlobe. The heart rate data may comprise instantaneous heart rate data.

The computer may be further configured to determine an effectiveness of an administered treatment using the estimated blood pressure and/or determine a condition of the subject based on the estimated blood pressure. Treatments may include those known in the art and may be those directly affecting blood pressure or other metrics of cardiovascular or heart health. Such treatments include but are not limited to pharmaceutical (including at least antihypertensive medications and diuretics) and surgical intervention. Treatments may include those known in the art and may be those directly affecting blood pressure or other metrics of cardiovascular or heart health including but not limited to hypertension, arrythmia, angina, heart attack, heart failure, valve disease, congenital heart defects, and others.

In some cases, the transmitted light signal is from a light-emitting diode (LED) source, laser, or combinations thereof. Other means of transmitting light signals are known in the art, some of which are described in the Examples. Data related to indicators of blood pressure may be acquired through means other than optical means of interrogation and can include bio-impedance, amongst others.

In some cases, the at least one indicator of blood pressure comprises SPG data, PPG data, and/or SCOS data. In at least cases wherein the at least one indicator of blood pressure comprises SPG data and/or PPG data, the light signals may be transmitted from an optical source and received by a photodetector that may be placed between about 1 mm and about 40 mm apart and/or the light signals may have wavelengths between about 300 nm and about 2000 nm. One of skill in the art will understand that light signals may be transmitted from an optical source and received by a photodetector that may be placed at distances not recited herein and can include 0 mm, 5 mm, 10 mm, 20 mm, 30 mm, 50 mm, and more. Additionally, one of skill in the art will understand that light signals may have wavelengths not recited herein and can include wavelengths less than 300 nm and greater than 2000 nm. In some cases, the optical source and/or detector are part of a wearable technology.

In at least cases wherein the at least one indicator of blood pressure comprises SPG data, the optical source may be coherent, and in some cases the level of coherence and source output power is determined by the separation of the source and detector. Coherence is a term commonly known in the art and can refer to the potential for two waves to interfere. In at least cases wherein the at least one indicator of blood pressure comprises PPG data, the light signal may be detected by a detector selected from the group comprising PIN photodiodes, avalanche photodiodes (APDs), single-photon avalanche diodes (SPADs), silicon photomultipliers (SiPM), photomultiplier tubes (PMTs), charge-coupled device (CCD), complementary metal-oxide-semiconductor (CMOS) camera sensors, and combinations thereof. In at least cases wherein the at least one indicator of blood pressure comprises SPG data the light signal may be detected by a detector selected from the group comprising single-photon avalanche diodes (SPADs), super conducting nanowire detectors (SNSPDs), silicon photomultipliers (SiPM), photomultiplier tubes (PMTs), high frame rate photodiodes, camera sensors, and combinations thereof. In at least cases wherein the at least one indicator of blood pressure comprises SCOS data, the light signal is detected by a detector selected from the group comprising photodiodes arrays, electron multiplying cameras, intensified cameras, standard CCD cameras, CMOS cameras, and combinations thereof.

In some embodiments of the invention, the detector is located on the head, neck or at least above the torso, for instance at or above where the neck attaches to the body (i.e., not below the head or not below where the neck attaches to the body). In certain embodiments, the detector may be located on the chest. In certain embodiments, the detector is not located on the extremities, for instance not on the finger, hand, arm, toe, foot, or leg.

In one example, PPG and/or SPG may be the signals to be measured and their time derivative may also be measured or determined. Devices, including light sources and detectors, may be used for this. For PPG, devices with LED and pin photodiodes may be used, including, for instance, NIRS devices, pulse oximeters, and cerebral oximeters. Measuring changes in detected light intensity may provide a suitable PPG signal. For SPG, a laser such as a long coherence length laser having a coherence length of, for instance, 1 m, 2 m, 3 m, 4 m, 5 m, 10 m, 100 m, 200 m, 300 m, or greater than 300 m may be used. Changes in speckle contrast may be used to provide the SPG signal. PPG may be also derived by a device that measures SPG by looking at the light intensity rather than the speckle contrast.

In another aspect, the present disclosure provides methods for estimating blood pressure, the method comprising: transmitting and receiving light signals from one or more locations on a subject using an optical coupling system configured to transmit and receive light signals; determining, using optical data generated using the received light signals using an optical processing system, at least one indicator of blood pressure; estimating blood pressure of the subject using the at least one indicator of blood pressure. The method may comprise utilizing any of the systems disclosed herein. In some cases, the method may further comprise generating a report indicative of the estimated blood pressure. The at least one indicator of blood pressure may comprise one or more of: near-infrared spectroscopy (NIRS) data; photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative data, second derivative

$$\frac{{d}^{2} P P G}{{dt}^{2}}$$

data, third derivative

$$\frac{{d}^{3} P P G}{{dt}^{3}}$$

data, first derivative

$$\frac{d S P G}{dt}$$

data, second derivative

$$\frac{{d}^{2} S P G}{{dt}^{2}}$$

data, inflow (Fᵢₙ) data, outflow (Fₒᵤₜ) data, heart rate data, physiological data, and combinations thereof. Further, the physiological data may comprise at least one of electrocardiogram (ECG) data, electroencephalogram (EEG) data, near infrared spectroscopy (NIRS) data, measured blood pressure data, respiratory data, hemoglobin data, pulse oximetry data, tissue oxygenation data, heart rate data, or a combination thereof. In some cases, the at least one indicator of blood pressure comprises at least PPG data and SPG data. In some cases, the at least one indicator of blood pressure comprises at least DCS data, NIRS data, and SCOS data. In some cases, the at least one indicator of blood pressure may comprise or further comprises a ratio of the magnitude of a pre-dicrotic peak (P₂) and a systolic peak (P₁) for at least one individual cardiac cycle in at least one of a PPG curve,

$$\frac{d P P G}{dt}$$

curve, an SPG curve, or a

$$\frac{d S P G}{dt}$$

curve. In some cases, the at least one indicator of blood pressure may comprise or further comprise one or more of subject height, subject weight, subject age, subject gender, subject body position data, subject location measured data and/or at a temporal resolution greater than a pulsatile frequency of the subject. In some cases, the pulsatile frequency is the pulsatile frequency of cerebral blood flow. The at least one indicator of blood pressure may be acquired during a portion of each cardiac cycle.

Estimated blood pressure may be at least one of systolic blood pressure (SBP), diastolic blood pressure (DBP), and mean arterial pressure (MAP).

The method may comprise performing a calibration against a measured blood pressure data set, and the measured blood pressure data set may be acquired by at least one of continuous measurements, non-continuous measurements, invasive (intra-arterial) blood pressure monitoring, non-invasive volume-clamp method (Finapres device, for example), cuff point measurements, automated oscillometry, manual auscultation, and combinations thereof.

The method may comprise applying a machine learning algorithm to estimate the estimated blood pressure by analyzing a measured blood pressure data set against at least one of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative

$$\frac{{d}^{2} P P G}{{dt}^{2}}$$

data, third derivative

$$\frac{{d}^{3} P P G}{{dt}^{3}}$$

data, first derivative

$$\frac{d S P G}{dt}$$

data, second derivative

$$\frac{{d}^{2} P P G}{{dt}^{2}}$$

data, inflow (Fᵢₙ) data, outflow (Fₒᵤₜ) data, heart rate data, physiological data, and combinations thereof. The machine learning algorithm approach may be at least one of random forests, support vector machines, gaussian process regression, and deep belief networks, and combination thereof. Means of applying a machine learning algorithm are known in the art and can comprise configuring a computer to apply said machine learning algorithms.

The one or more locations on a subject may comprise the subject's forehead, neck or earlobe. In some embodiments the one or more locations on a subject may comprise the subject's chest. The heart rate data may comprise instantaneous heart rate data.

The method may further comprise determining an effectiveness of an administered treatment using the estimated blood pressure and/or determining a condition of the subject based on the estimated blood pressure.

In some cases, the transmitted light signal is from a light-emitting diode (LED) source, other diode, laser, or combinations thereof.

In some cases, the at least one indicator of blood pressure comprises SPG data, PPG data, and/or SCOS data. In at least cases wherein the at least one indicator of blood pressure comprises SPG data and/or PPG data, the light signals may be transmitted from an optical source and received by a photodetector that may be placed between 1 mm and 40 mm apart and/or the light signals may have wavelengths between about 300 nm and about 2000 nm. In at least cases wherein the at least one indicator of blood pressure comprises SPG data, the optical source may be coherent, and in some cases the level of coherence and source output power is determined by the separation of the source and detector. In at least cases wherein the at least one indicator of blood pressure comprises PPG data, the light signal may be detected by a detector selected from the group comprising PIN photodiodes, avalanche photodiodes (APDs), single-photon avalanche diodes (SPADs), silicon photomultipliers (SiPMs), photomultiplier tubes (PMTs), charge-coupled device (CCD), complementary metal-oxide-semiconductor (CMOS) camera sensors, and combinations thereof. In at least cases wherein the at least one indicator of blood pressure comprises SPG data the light signal may be detected by a detector selected from the group comprising single-photon avalanche diodes (SPADs), super conducting nanowire detectors (SNSPDs), silicon photomultipliers (SiPMs), photomultiplier tubes (PMTs), high frame rate photodiodes, camera sensors, and combinations thereof. In at least cases wherein the at least one indicator of blood pressure comprises SCOS data, the light signal is detected by a detector selected from the group comprising photodiodes arrays, electron multiplying cameras, intensified cameras, standard CCD cameras, CMOS cameras, and combinations thereof.

In some embodiments of the invention, the detector is located on the head, neck or at least above the torso, for instance at or above where the neck attaches to the body (i.e., not below the head or not below where the neck attaches to the body). In certain embodiments, the detector may be located on the chest. In certain embodiments, the detector is not located on the extremities, for instance not on the finger, hand, arm, toe, foot, or leg.

The systems and methods disclosed herein may be applied to human and/or non-human subjects and/or patients.

The present disclosure illustrates at least the following innovations over the prior art. Specific innovations at least include the use of speckle imaging features (e.g. the P2/P1 feature) and the need for a placement of the probe on the forehead, neck, and/or earlobe. Specific innovations also at least include the use of the PPG derivative signal and the specific features derived from those signals. Specific innovations also at least include the use of beat shape as opposed to pulse transit time. Specific innovations also at least include the use of time domain features over frequency domain features in the derivation of pressure.

It will be appreciated by those skilled in the art that while the disclosed subject matter is described herein in connection with particular embodiments and examples, the disclosure is not necessarily so limited and that numerous other embodiments, examples, uses, modifications and departures from the embodiments, examples, and uses are intended to be encompassed by the claims attached hereto.

Various features and advantages of the invention are appreciated by one with skill in the art, some of which are set forth in the present disclosure. The number of possibilities for innovation in this field remains nearly infinite while the guidance from successful examples is practically zero. Because of this, Applicant submits that the bar for what might be considered inventive in this space needs to properly consider these factors.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Measuring Blood Pressure Non-Invasively with Light

In certain embodiments, blood pressure estimation involves measuring an optical waveform

$$( P P G , \frac{d P P G}{dt} \frac{{d}^{2} P P G}{{dt}^{2}} , or S P G , or P P G , \frac{d P P G}{dt} \frac{{d}^{2} P P G}{{dt}^{2}} \& S P G )$$

and analyzing the shape of the measured curve. We can also use curves of inflow and outflow (Fᵢₙ and Fₒᵤₜ) derived from the measured PPG and SPG curves as inputs to the model. In one instance, the ratio of the magnitude of P₂ and P₁ for individual cardiac cycles in either the

$$\frac{d P P G}{dt}$$

or SPG curves is calculated. The instantaneous heart rate during each cardiac cycle is estimated as the inverse of the duration of the cardiac cycle. Estimations of blood pressure can then be found by performing multivariate linear regression, for example by fitting a linear model of the form

$$B P = {c}_{1} + {c}_{2} * \frac{{P}_{2}}{{P}_{1}} + {c}_{3} * H R .$$

In other instances, the normalized optical waveforms are passed through machine learning approaches to generate estimates of blood pressure. Other parameter of interest to optimize the estimation of blood pressure are subject height, weight, age, gender, body position and location measured. These models can be used to estimate the systolic blood pressure (SBP), diastolic blood pressure (DBP), and/or the mean arterial pressure (MAP). Currently, this method relies on a calibration period where both optical measurements and blood pressure measurements are taken concurrently. The blood pressure measurement could include continuous measurements, like invasive (intra-arterial) blood pressure monitoring or the non-invasive volume-clamp method (Finapres device, for example), or point measurements, like automated oscillometry or manual auscultation.

For both SPG and PPG measurements, an optical source and photodetector are placed some distance apart, typically between 1 mm and 40 mm. The wavelength can be any between 300-2000 nm. The source for PPG measurements can be either a light emitting diode (LED) or a laser. The source for SPG measurements must be coherent (e.g. a laser diode), though the required level of coherence and source output power is determined by the separation of the source and detector. For example, for short separation measurements (˜5 mm), a laser with lower power output and a lower coherence length could be used. For PPG, possible classes of detectors include, though are not limited to, PIN photodiodes, avalanche photodiodes (APDs), photomultiplier tubes (PMTs), and charge-coupled device (CCD) or complementary metal-oxide-semiconductor (CMOS) camera sensors. For SPG measured by DCS, the possible classes of detectors include, though are not limited to, single-photon avalanche diodes (SPADs), super conducting nanowire detectors (SNSPDs), PMTs, and high frame rate (>300 kHz) photodiode or camera sensors when DCS heterodyne detection is utilized. For SPG measured by SCOS, the possible classes of detectors include, though are not limited to, photodiodes arrays, electron multiplying or intensified or standard CCD cameras and CMOS cameras. The light coming from the source and going into the detector can either be coupled by putting the source and detector components directly on the skin, coupling the light in free-space through the use of lenses and other optical components, coupling the light into optical fibers, or any combination of the above methods could be used. The sensor consisting of source and detector is placed on the surface of the skin at some location on the body, including but not limited to, the forehead, wrist, fingertip, and/or earlobe.

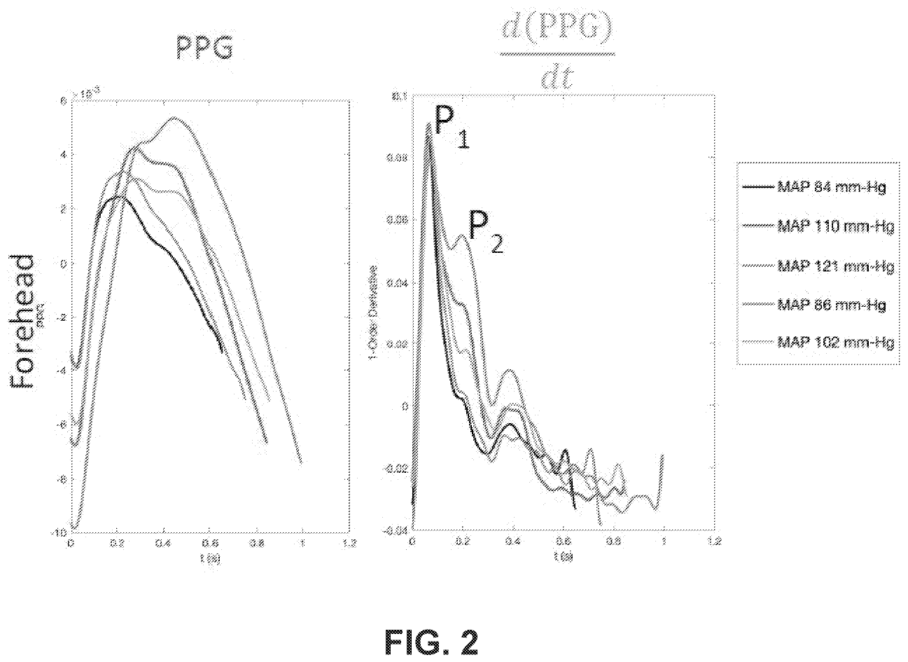

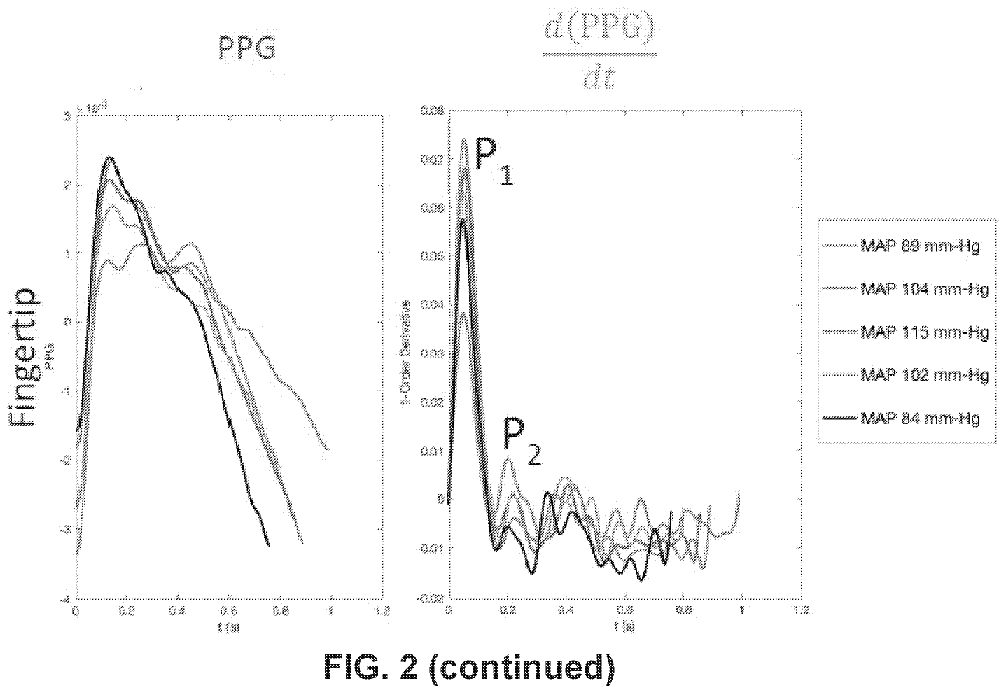

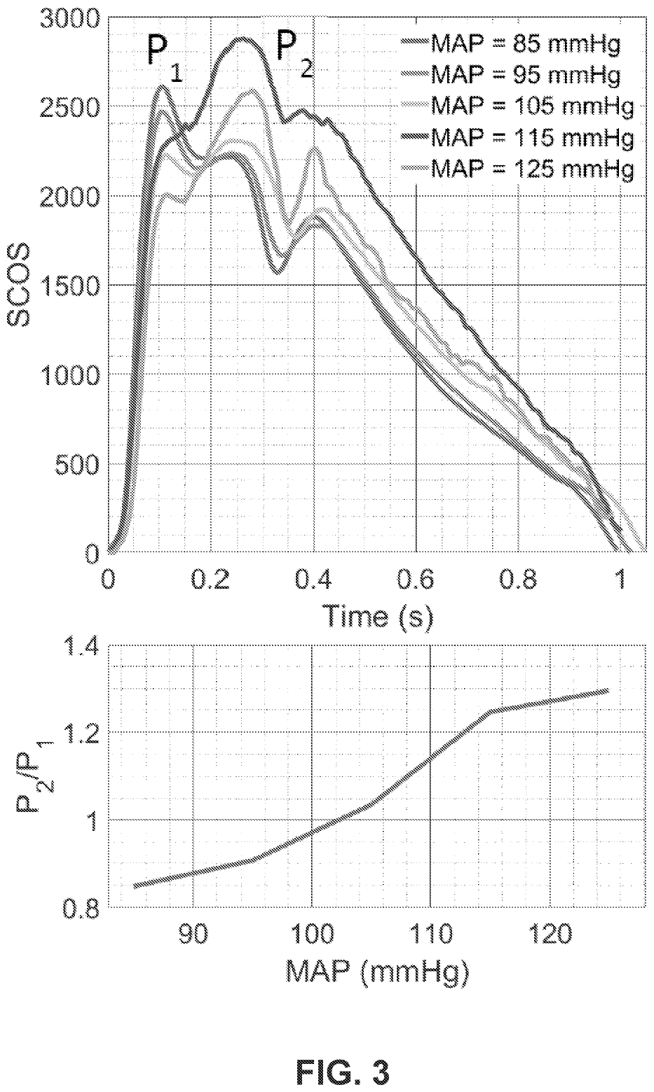

Examples of the morphology of the PPG signal at different body locations and different mean arterial pressure (MAP) can be seen in FIG. 2. These results indicate some positions on the body are less useful for optical blood pressure monitoring by this method due to the lack of a consistent P₂ feature (fingertip). An example of a measurement of the SPG waveform collected from the earlobe at different MAPs can be seen in FIG. 3. The relationship between the ratio of P₂ and P₁ is also plotted to show that the change in the ratio is associated with the changes in mean arterial pressure. Utilizing the ratios extracted from the

$$\frac{d P P G}{dt}$$

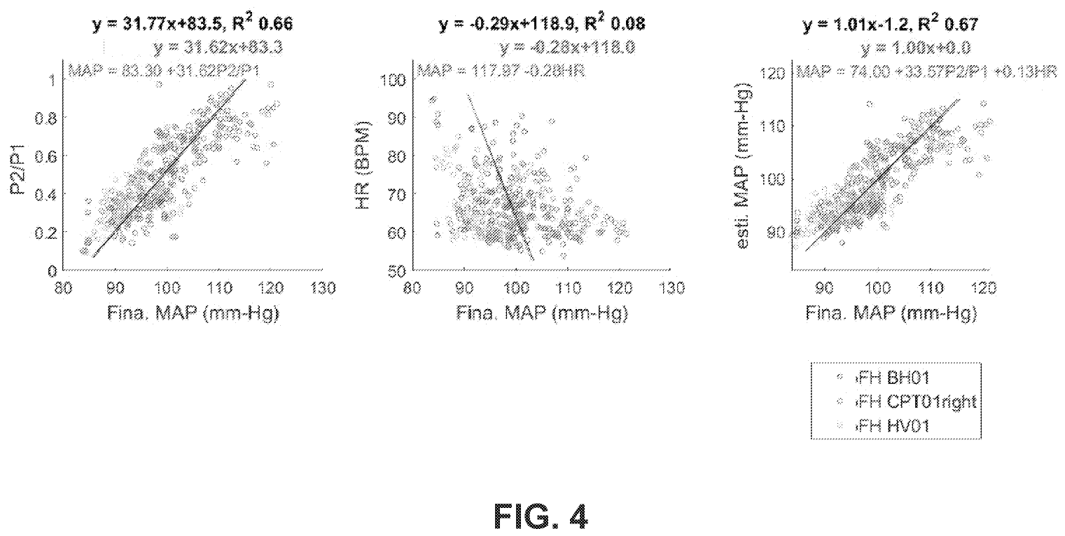

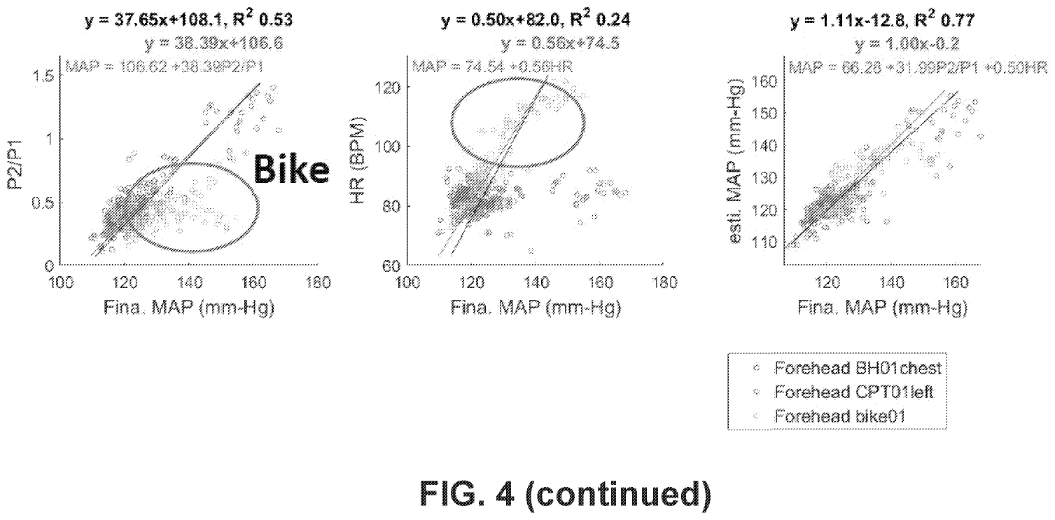

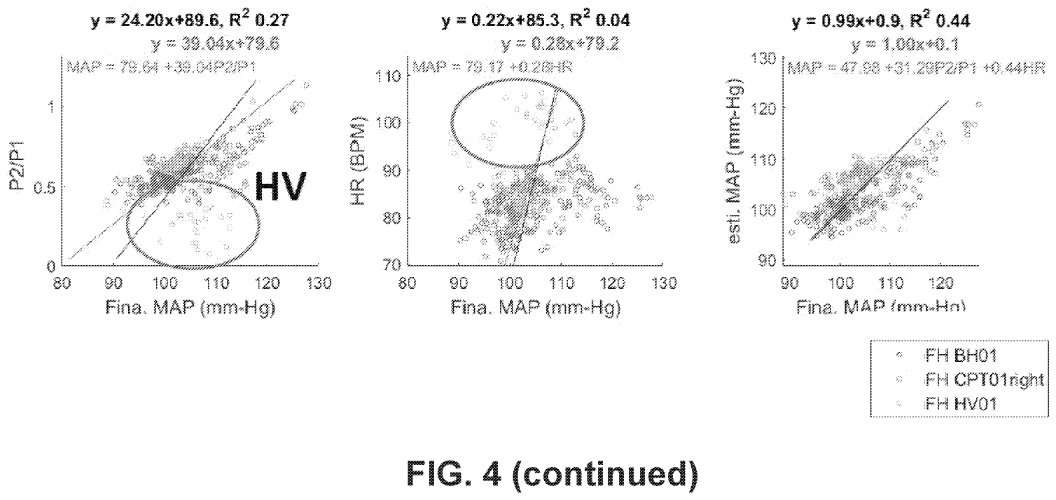

curves and/or the SPG curves along with the heart rate, blood pressure parameters can be estimated. In FIG. 4, three examples of the use of the multivariate linear model with PPG datasets collected on the forehead are shown. The linear model is fit individually with peak ratio data (P₂/P₁), heart rate data, and a combination of the variables to show the importance of the inclusion of both the ratio of the peak magnitude and the heart rate. A significant deviation of the fit of MAP can be seen in the fitting where only the ratio of the peaks is used in the regions labeled “Bike” (data collected while the subject was biking) and “HV” (data collected while the subject was hyperventilating), where it was observed that the shape of the

$$\frac{d P P G}{dt}$$

curve did not substantially change with increasing blood pressure, though the heart rate did. The inclusion of the heart rate in the analysis allows for more accurate characterization of the blood pressure.

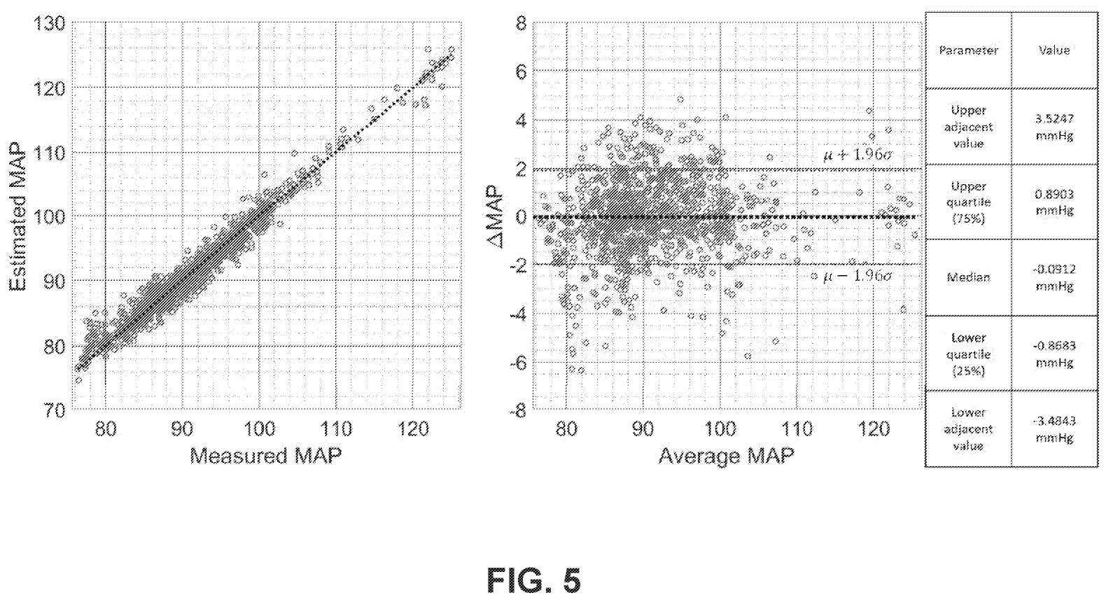

The application of machine learning approaches in the estimation of blood pressure from measurements made by these non-invasive methods presents an opportunity to improve the accuracy of blood pressure recovery as well as biomarker discovery in determining features in the shapes of the measured curves that are sensitive to changes in blood pressure. An example of the performance of one of these methods is shown in FIG. 5. Taking inputs of individual SPG curves at different MAP values and the heart rate for each cardiac cycle, gaussian process regression was used to estimate the relationship between changes in the shape of the SPG curve and the mean arterial pressure. As seen in the Bland-Altman plot, the 95% confidence interval for the estimation of MAP is ±3.12 mmHg for this data set, showing excellent agreement with the measured blood pressure. The disclosed methods and systems are distinct from existing methods in a number of ways at least including the P₂ and P₁ optical data/features used as inputs, the inclusion of heart rate as input parameter, and the combination of SPG and PPG data collection. Specific innovations over prior art at least include the use of speckle imaging features not explicitly mentioned in the patent (e.g. the P₂/P₁ feature) and the need for a placement of the probe on the forehead or earlobe. Also novel is the use of the PPG derivative signal and the specific features derived from those signals. The mentioned machine learning approaches are random forests, support vector machines, and deep belief networks, which do not overlap with our current expectations of possible machine learning classes used. Also novel is the use of beat shape as opposed to pulse transit time, which almost completely reduces overlap between the inventions. Also novel is the use of time domain features over frequency domain features in the derivation of pressure. The difference of estimating ICP as opposed to blood pressure can be significant.

A summary of new features/innovations added by our methods is provided below:
a. Use of new feature (P₂/P₁) from the time derivative of the PPG signal and/or SPG signal
b. Use of the combination of SPG and PPG
c. Calculation of vascular in-flow and out-flow (with the model described in PCT WO2016164894) using the combination of simultaneously acquired SPG and PPG signals to derive a signal that is more closely related to arterial blood flow
d. Use of the instantaneous heart rate as a parameter in blood pressure estimation
e. Use of SCOS, which at short source-detector separations (high SNR), can estimate the blood pressure on a beat to beat basis
f. Use of AI methods with the entire shape of the PPG, its time derivatives, and/or SPG curves to estimate blood pressure

### Example 2: Enhancing Diffuse Correlation Spectroscopy Pulsatile Cerebral Blood Flow Signal with Near-Infrared Spectroscopy Photoplethysmography

Combining near-infrared spectroscopy (NIRS) and diffuse correlation spectroscopy (DCS) allows for quantifying cerebral blood volume, flow, and oxygenation changes continuously and non-invasively. As recently shown, the DCS pulsatile cerebral blood flow index (pCBFi) can be used to quantify critical closing pressure (CrCP) and cerebrovascular resistance (CVRi). While current DCS technology allows for reliable monitoring of the slow hemodynamic changes, resolving pulsatile blood flow at large source-detector separations, needed to ensure cerebral sensitivity, is challenging because of its low signal-to-noise ratio (SNR). Cardiac-gated averaging of several arterial pulse cycles is required to obtain a meaningful waveform. Taking advantage of the high SNR of NIRS, we demonstrate a new method that uses the NIRS photoplethysmography (NIRS-PPG) pulsatile signal to model pCBFi, reducing the coefficient of variation of the recovered pulsatile waveform (pCBFi-fit), allowing an unprecedented temporal resolution (266 Hz) at a large source-detector separation (>3 cm). In 10 healthy subjects, we verify the quality of the NIRS-PPG pCBFi-fit during common tasks, showing high fidelity against pCBFi (R2 0.98±0.01). We recover CrCP and CVRi at 0.25 Hz, >10 times faster than previously achieved with DCS. NIRS-PPG improves DCS pCBFi SNR, reducing the number of gate-averaged heartbeats required to recover CrCP and CVRi.

The following Example describes a system and setup designed to measure intracranial pressure. The Example is intended to serve the skilled practitioner at least the foundational knowledge of concepts important to practice the invention. The Example is not intended to be a limiting embodiment of the invention.

### Introduction

Near-infrared spectroscopy (NIRS) is an established non-invasive diffuse optical method used to quantify hemoglobin oxygen saturation (SO2) and hemoglobin concentration (Hb) changes from light attenuation at different wavelengths (1,2). Diffuse correlation spectroscopy (DCS) is a more recent optical technology which uses speckle fluctuations to measure an index for blood flow (BFi). Specifically, DCS measure the intensity temporal autocorrelation function, (g2) which characterize the displacement dynamics of scattering particles in the sample under interrogation (3-5). Combining DCS with NIRS allows for calculating the cerebral metabolic rate of oxygen (6) and furthering our understanding of the relationship between hemoglobin concentration and cerebral blood flow (CBF) changes in healthy (7,8) and pathological conditions (9,10). More recently, our and other groups have proposed the use of DCS pulsatile cerebral blood flow index signals (pCBFi) to quantify intracranial pressure (ICP), critical closing pressure (CrCP), pulsatility index (PI) and cerebrovascular resistance index (CVRi) continuously and non-invasively (11-16). While DCS alone is promising, the low signal-to-noise ratio (SNR) of current DCS devices limits pCBFi to source-detector separations (SDsep) of up to 2.5 cm which provides low brain sensitivity in adults (17), and to achieve sufficient time-points within a pulsatile waveform, requires cardiac-gate averaging of 50 arterial pulses (11), which dampens the pulsatile peaks and provides CrCP and CVRi estimates at 0.02-0.07 Hz, rates that are too low to investigate the dynamic pressure-flow relationship of the cerebral vasculature (18). To overcome the DCS noise, increase SDsep, and recover pCBFi with much less averaging, we propose a new method based on the combination of NIRS and DCS pulsatile signals.

Because NIRS measurements at the same sampling rate typically detected multiple orders of magnitude more photons, the SNR of NIRS is much better than the SNR of DCS (19,20), allowing for the measurement of pulsatile blood volume waveforms with high temporal resolution at long SDsep (≥3 cm) (21). In particular, an open-source, novel, wearable, wireless NIRS device that we recently developed, FlexNIRS, is capable of acquiring data from four LED sources and three detectors at 266 Hz (originally 100 Hz) with a very low noise equivalent power (NEP<70 fW/√Hz) (22). The high SNR performance of this device allows us to resolve the pulsatile blood volume and its time derivative to perform cerebral pulse waveform analysis (PWA) on the pulsatile light absorbance of NIRS photoplethysmography (PPG) at a 3.3 cm SDsep (NIRS-PPG) with zero to a few beats averaging. PWA generally refers to the study of the morphology of the PPG waveform measured at short SDsep with pulse oximetry devices (23). Morphological features extracted from the superficial PPG and its time derivative have been investigated in the literature and often include amplitude, latency and width of the PPG wave contour. These features are generally located with algorithms that finds the local maximum and minimum in the signals and its 1st to 3rd time derivatives (23). PWA quantifies pulse wave characteristics to obtain information on cardiovascular state, and correlations of specific features with skin blood vessel aging, stiffness and peripheral resistance have been demonstrated (24-27). The ability to measure long SDsep PPG and its time derivative extends the analysis to characterize cerebral blood vessels and opens a new dimension to studying brain health with diffuse optical methods (28-30). Further, by exploiting the pulsatile blood volume and blood flow relationship (31-33), we can separate the pulsatile inflow and outflow, and model pCBFi as a linear contribution of NIRS-PPG and its first time derivative (d (NIRS-PPG)/dt). The resulting fitted pulsatile cerebral blood flow (pCBFi-fit) displays exceeding SNR over DCS, while accurately matching DCS pulsatile flow, allowing us to estimate PI, CrCP and CVRi at the cardiac frequency.

To validate this model, we measured 12 healthy subjects simultaneously with the FlexNIRS and a state-of-the-art DCS prototype available in our lab, which operates at 1064 nm and employs superconducting nanowire single-photon detectors (SNSPD). The SNSPD-DCS system provides a >16-fold SNR increase vs. the standard DCS technology (17), allowing us to resolve pCBFi at larger separations and to use lower cardiac-gated averaging. We performed the NIRS and DCS measurements on the subjects while performing standard tasks that change cerebral and systemic physiology and recovered both pulsatile and slow varying signals under various conditions.

### Material and Methods

### Participants and Measurement Protocol

For the study, we enrolled and measured 12 healthy subjects. Two subjects were excluded due to low DCS data quality: low DCS photon counts (<20 kcps/s) and low coherence factor (Siegert relationship β<0.3) 34. Data from these two subjects were not included in this paper and were not numbered. The remaining 10 subjects included 6 males and 4 females of diverse skin tones, with an age range of 21 to 68 years old (Table 1). The study protocol was reviewed and approved by the Mass General Brigham Institutional Review Board (IRB #2020P002463) in accordance with Ethical Principles and Guidelines for the Protection of Human Subjects (Belmont Report). All subjects signed the informed consent before participating in the study.

The protocol consisted of cold pressor tests (CPT), paced hyperventilation (HV) and brief repeated breath-holding (BH) tasks. For the CPT, subjects submerged one hand in warm water (30° C.-36° C.) for two minutes; then they moved the hand into icy cold water (3° C.-5° C.) for one minute; finally, they moved the hand back into the warm water for two more minutes. CPT is known for raising blood pressure (35), and the protocol barred any subject with high blood pressure from performing this task. For the paced HV, after a one-minute baseline of sale-paced breathing, subjects breathed at a rate of 70 breaths per minute following a metronome cue for a minute, and then resumed their self-paced breathing for two more minutes. For the BH task, after a one-minute baseline, subjects repeated a set of 20 s breath-holding and 40 s normal breathing for four times, ending with an extended recovery period of one minute.

TABLE 1
Participant demographic information.

| | | | | Height | Weight | |
|---|---|---|---|---|---|---|
| New | Age | Gender | BMI | cm | kg | Skin tone |
| sub01 | 23 | Male | 20.0 | 168 | 56 | Light |
| sub02 | 24 | Male | 28.2 | 170 | 82 | Light |
| sub03 | 63 | Female | 25.1 | 173 | 75 | Light |
| sub04 | 68 | Male | 29.6 | 170 | 86 | Light |
| sub05 | 40 | Male | 19.5 | 180 | 63 | Light |
| sub06 | 23 | Female | 22.9 | 157 | 57 | Dark |
| sub07 | 36 | Male | 24.3 | 165 | 66 | Medium |
| sub08 | 55 | Female | 28.2 | 170 | 82 | Dark |
| sub09 | 26 | Female | 25.4 | 158 | 64 | Light |
| sub10 | 21 | Male | 25.8 | 168 | 73 | Light |
| Avg ± | 38 ± | 4F, 6M | 24.9 ± | 167.9 ± | 70.4 ± | 7 L, 1 M, |
| SD | 17 | | 3.2 | 6.4 | 10.2 | 2 D |

For the skin tone classification, we used the Fitzpatrick classification of skin types regrouped as: Light: pale white skin, white fair skin (types 1 and 2); Medium: medium white skin, olive, light brown skin (types 3 and 4); Dark: brown skin, dark brown, or black skin (types 5 and 6). (L: light, M: medium, D: dark)

### Devices

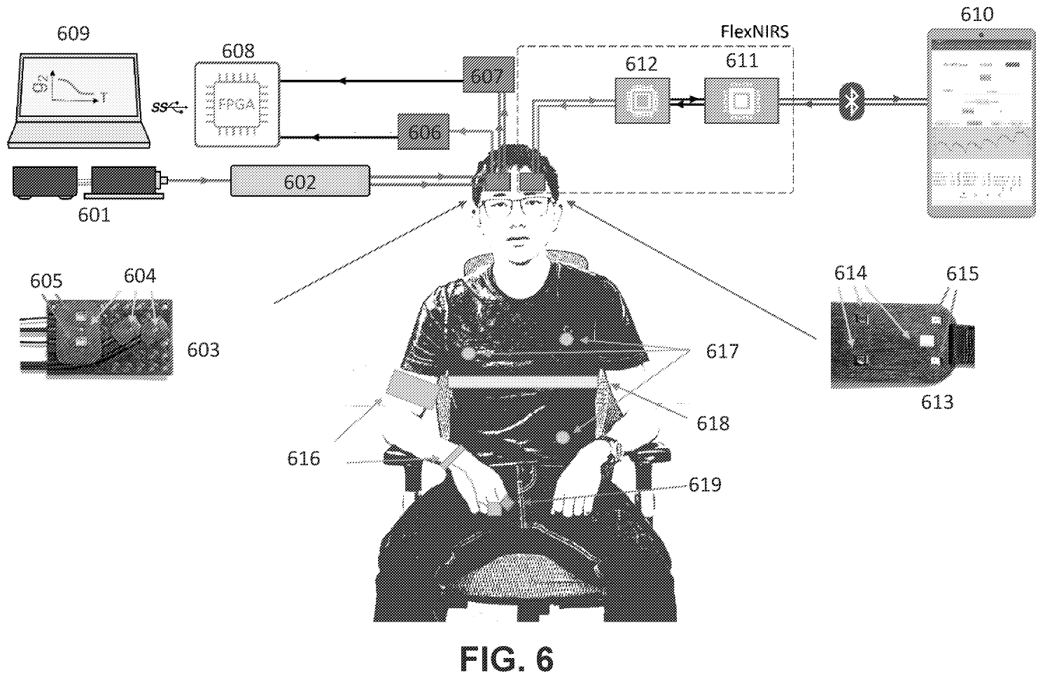

The wearable NIRS system used in the study is a continuous wave, open-source, low-cost, wearable wireless cerebral oximeter called FlexNIRS, recently developed in our group (22). It consists of a wearable NIRS headband with an embedded flexible optical probe, communicating with a tablet through Bluetooth low energy (BLE) and is powered by a rechargeable lithium-ion battery (FIG. 6). The probe includes two dual-wavelength LEDs (735 and 850 nm, SMT735D/850D, Marubeni, Japan) and three PIN-photodiodes (VEMD5060X01, Vishay Intertechnology, Inc., USA), forming 2 short SDsep of 0.8 cm and 2 symmetrically arranged long SDsep of 2.8 cm and 3.3 cm. This geometry allows for a self-calibrating multi-distance scheme, which by using two values at each separation eliminates the effect of different component efficiencies, and therefore provides a robust measurement of cerebral hemoglobin oxygenation without the need for external calibration (36,37). The use of surface-mount source and detector components in direct contact with the skin minimizes signal attenuation, and the use of a dedicated analog front-end chip that amplifies and digitizes the signal on one integrated circuit results in a device with low NEP (<70 fW/VHz). The optimized custom software allows for a sampling rate of 266.66 Hz for all channels. A greater than 2.5 times improvement in sampling rate over the original description of the FlexNIRS device (100 Hz) (22). This was achieved through shortening the sampling durations (LED-on time for all was reduced from 366 to 117 μs) and eliminating repeated ambient and short SDsep channels.

The DCS system used in this study operates at 1064 nm using a long coherence length laser (CrystaLaser, USA) and four high photon detection efficiency (88%) SNSPD detectors (Opus One, Quantum Opus, USA) 17 (FIG. 6). To further increase SDsep while maintaining SNR, we used a dual-source illumination scheme, splitting the laser into two 3.5 mm diameter illumination spots each emitting up to the maximal permissible exposure limit (MPE) of 103 mW at 1064 nm (ANSI Z136.1). Source and detector fibers were terminated with prisms into a custom optical probe made of flexible 3D-printing material (NinjaTek, USA) and arranged to form SDsep of 2.0 cm (one detector fiber) and 3.0 cm (three detector fibers) (FIG. 6). In addition, to acquire scalp signals, we used a silicon single-photon avalanche diode (Si SPAD) detector at 0.5 cm from the sources. A custom 20-channel field-programmable gate array (FPGA) board documented the arrival time of each photon at 150 MHz resolution, and a custom data acquisition software calculated and displayed the g2 and the blood flow index for each channel in real-time.

To avoid possible crosstalk between FlexNIRS and the DCS, we positioned the two probes symmetrically one on opposite sides of the forehead with the source placed laterally to maximize the distance between them.

We monitored systemic physiology, using a non-invasive continuous arterial blood pressure (ABP) monitor (Finapres Medical Systems, Netherlands; Finapres NOVA) and BIOPAC modules (BIOPAC Systems Inc., USA) for electrocardiogramalse oximetry peripheral oxygen saturation (SpO2; OXY100E) and respiratory rate (SS5LB). All systemic physiological signals were acquired with a 1000 Hz digital data acquisition device (PowerLab, ADInstruments, New Zealand).

### Data Analysis

FlexNIRS and DCS data were analyzed using standard routines to recover slow varying hemodynamic changes and with cardiac-gated averaging algorithms to extract pulsatile waveforms.

To estimate slow varying blood flow changes, we computed the g2 curves at 1 Hz smoothed with a moving average of 3 s for each SDsep. The g2 obtained by 3 colocalized detectors at 3.0 cm were averaged together to increase SNR. We calculated BFi by fitting the g2 to the semi-infinite correlation diffusion equation 40, using optical properties μs' of 4.7 cm-1 and μa of 0.18 cm-1. The scattering was extrapolated from the values used for FlexNIRS at shorter wavelengths to 1064 nm, and the absorption was obtained assuming typical hemoglobin and water concentrations (41).

In addition, we calculated heart rate (HR) as the inverse of the R-R interval in the ECG signals and extracted time series of ABP from calibrated Finapres measurements and SpO2 from the pulse oximeter. For group averaging, optical and systemic measured parameters were down-sampled to 1 Hz, subjected to a 3-s window moving average, and normalized with respect to the initial baseline. The four repetitions of BH were averaged together after detrending and normalizing the data to the three-second periods before the BH. Before averaging across subjects, the missing points that were rejected due to motion artifacts were interpolated.

### Pulsatile Waveform Analysis

To extract pulsatile waveforms from the FlexNIRS data, we first subtracted ambient light from the raw intensity measured at 266 Hz and removed data segments with motion artifacts as detected by the embedded accelerometer and gyroscope. We used a high pass filter (fc=0.4 Hz) to remove breathing and Mayer waves and a low pass filter (fc=14 Hz) to remove high-frequency noise. We then calculated the delta absorbance, or delta optical density (ΔOD=In [IO/I (t)], IO: baseline light intensity) 42 at 850 nm. In addition to motion artifacts, we rejected distorted heartbeats that had outliers, which were defined as data points deviating from the median waveform by more than 2.5 interquartile range (IQR). The median and IQR were calculated with cardiac-gated heartbeats within a short moving window of 15 s with a 50% overlap. To establish condition-representing NIRS-PPG waveforms, we gate-averaged heartbeats measured during the various tasks (conditions): Each one-minute baseline was considered as one condition; the same was done for each recovery segment, excluding the first 20-s transition period; during the hyperventilation and cold pressor tests, we excluded the initial transitions and considered the last 40 s with more stable hemodynamic responses; for the breath-holding condition, we considered a period of 20 s centered around the end of the BH, and averaged the four repetitions together. The FlexNIRS optical data were aligned with the ECG signals by matching the beat-to-beat heart rate variability. For the gate averaging, the cardiac cycles were identified using the diastolic onsets in the NIRS-PPG signals and each heartbeat was normalized by stretching, through interpolation, to a uniform period equivalent to the subject average heart rate during that condition. While we performed these calculations for both NIRS wavelengths and all source-detector pairs, for each subject NIRS-PPG PWA analysis we report the 3.3 cm pair at 850 nm with the highest SNR (SNR=σpulsation/σnoise; σ: standard deviation). The SNR was calculated based on high-pass filtered light intensity signals (fc=0.4 Hz): the pulsation is defined as the low-pass filtered (fc=14 Hz) intensity, whereas the noise is the difference between pre and post low-pass filtering. In two subjects (#3 and #8), a 2.8 cm channel was used because the 3.3 cm channels had low SNR (<15).

To obtain DCS pulsatile blood flow waveforms, we computed g2 curves at 100 Hz using 40 ms of integration time, i.e. a 20 ms photon-inclusion radius. The data periods excluded in the FlexNIRS due to motion artifacts were also excluded for the DCS time series. Representative DCS pulsatile blood flow waveforms during the various conditions were calculated through cardiac-gated averaging of g2 curves instead of the CBFi. Each heartbeat onset was determined by the co-acquired ECG R-peaks; across heartbeats, g2 curves of the same location in the cardiac cycle were averaged together as done in a previous study 11. As for the slow CBFi, the gate-averaged g2 curves were fitted using the semi-infinite correlation diffusion equation 40 assuming the optical properties listed previously. During the fitting iterations for the pCBFi, as systolic g2 curves of high flow lack a clear plateau, β was kept constant. For each measurement, this value was set to be the median β from the traditional free-β fit.

Pulse waveform analysis was performed to quantify the peak amplitudes and their corresponding latencies of NIRS-PPG, d (NIRS-PPG)/dt and pCBFi. We used a time derivative method (23) modified for brain PPG. When comparing pulsatile NIRS and DCS, to match the NIRS sampling rate, pCBFi was up-sampled to 266 Hz through cubic spline interpolation; to compensate for the longer integration time of DCS (40 ms), NIRS-PPG and d (NIRS-PPG)/dt were subjected to a moving average of 11 points (i.e. 11/266.66 Hz≈40 ms).

Modeling of pCBFi as a Function of NIRS-PPG.

The pulsatile light absorbance of NIRS-PPG is proportional to the pulsatile blood volume changes. As a result, its first time derivative represents the changing rate of blood volume, which is the difference between the blood inflow and the blood outflow during an heartbeat 31-33:

$${BF}_{in} - {BF}_{out} = γ \frac{d}{dt} ( NIRS - PPG ) \qquad ( 1 )$$

As suggested by a prior study, if we assume the outflow blood drains out passively, BFout can be modeled to be proportional to the volume of blood within the tissue bed. This is analogous to assuming a blood volume exponential decay, as indicated by the first-order differential equation (dV/dt=−BFout=−α·V; V: blood volume, α: constant) 31.

$${BF}_{out} = a ( NIRS - PPG ) + b \qquad ( 2 )$$

DCS pCBFi represent the total flow through the vasculature, which gives us:

$${BF}_{in} + {BF}_{out} = k \cdot {pCBF}_{i} \qquad ( 3 )$$

with k being the scaling factor that converts pCBFi (cm/s2) to blood flow units (mL/(100 g*min)) for perfusion per tissue weight.

By combining these equations, we can derive pCBFi-fit, the estimated pCBFi based on the linear combination of the NIRS-PPG signal and its derivative:

$$\begin{matrix}{pCBF}_{i - fit} = \frac{γ}{κ} \frac{d}{dt} ( NIRS - PPG ) + \frac{2 a}{κ} ( NIRS - PPG ) + \frac{2}{κ} b \\ = {C}_{1} \frac{d}{dt} ( NIRS - PPG ) + {C}_{2} ( NIRS - PPG ) + {C}_{3}\end{matrix} \qquad ( 4 )$$

An additional parameter, the alignment lag C0 was applied to temporally align the signals acquired with independent devices. This is due to the uncertainty of absolute time bases of the devices introduced by the BLE communication and the clocks of the tablet and the computer.

A non-linear fit, Levenberg-Marquardt with sweeping initial guess, was performed to minimize the residuals and determine the four parameters (C0, C1, C2 and C3).

Finally, by combining Eq. (1), (3) and (4), we were able to separate the inflow (BFin) and outflow (BFout) contributions to the pulsatile flow:

$${BF}_{in} = \frac{1}{2} ( pCBFi + {C}_{1} \frac{d}{dt} ( NIRS - PPG ) ) \qquad ( 5 )$$

$${BF}_{out} = \frac{1}{2} ( pCBFi - {C}_{1} \frac{d}{dt} ( NIRS - PPG ) ) \qquad ( 6 )$$

We calculated C1 and C2 for each task condition by fitting the averaged pCBFi and NIRS-PPG signals, and then projected these parameters back to NIRS-PPG to estimate pCBFi-fit with no cardiac-gated averaging. C1 and C2 were interpolated during task-transitions. C3 was replaced by the slow varying mean CBFi time traces calculated above. We evaluated the SNR improvement with respect to SNSPD-DCS pCBFi, by calculating the coefficient of variation (CV) as σ/μ (σ: standard deviation, μ: mean) of each point along the cardiac cycle across heartbeats during the 60-s baselines.

### Critical Closing Pressure, Pulsatility Index and Cerebrovascular Resistance.

We obtained the Pl, CrCP and CVRi using the pCBFi and the alternative NIRS-PPG-reconstructed pCBFi-fit with much less cardiac-gated averaging. With the SNSPD-DCS-only pCBFi, due to its higher noise, we used 20 heartbeats; with NIRS-PPG-reconstructed pCBFi-fit, we used 4 heartbeats. The signals were aligned with the pulsatile ABP (pABP) obtained from the Finapres using the same cardiac-gated averaging. CrCP was obtained by linearly extrapolating the pCBFi vs. pABP relationship to the pABP-axis intercept. Because of the non-linearities during the systolic phase, only the diastolic run-off phase of the pulse cycle was considered as done previously 11. In the past, the dicrotic notch was manually found for each subject; however, now thanks to the high SNR of the pCBFi-fit, we can automatically find the diastolic run-off using a PWA algorithm (23) which we customized for brain NIRS-PPG. The cerebrovascular resistance index was calculated as the inverse of the slope of the pulsatile pressure-flow relationship:

$${CVR}_{i} = \frac{d ( pABP )}{d ( {PCBF}_{i} )} \qquad ( 7 )$$

We rejected outliers with loose criteria that only include data points that have pressure-flow regression R2 of higher than 0.8, CrCP within-30 to 150 mmHg and positive CVRi.

The pulsatility index (PI), defined as the difference between the systolic and diastolic flow velocity divided by the mean flow velocity as measured by transcranial Doppler ultrasound (TCD), is used to assess vascular resistance (43), intracranial pressure and cerebral perfusion pressure (44). Herein we define PI of DCS as the difference between the systolic and diastolic pCBFi divided by the mean. The same was calculated for pCBFi-fit.

When comparing results between conditions, after excluding outliers (exceeding 1.5 IQR above Q3 or below Q1), we examined normality with the Kolmogorov-Smirnov test (α=0.05). If normality was not found, instead of the pairwise t-test, we applied paired-sample sign test.

### Results

### Pulsatile CBFi and NIRS-PPG Waveforms

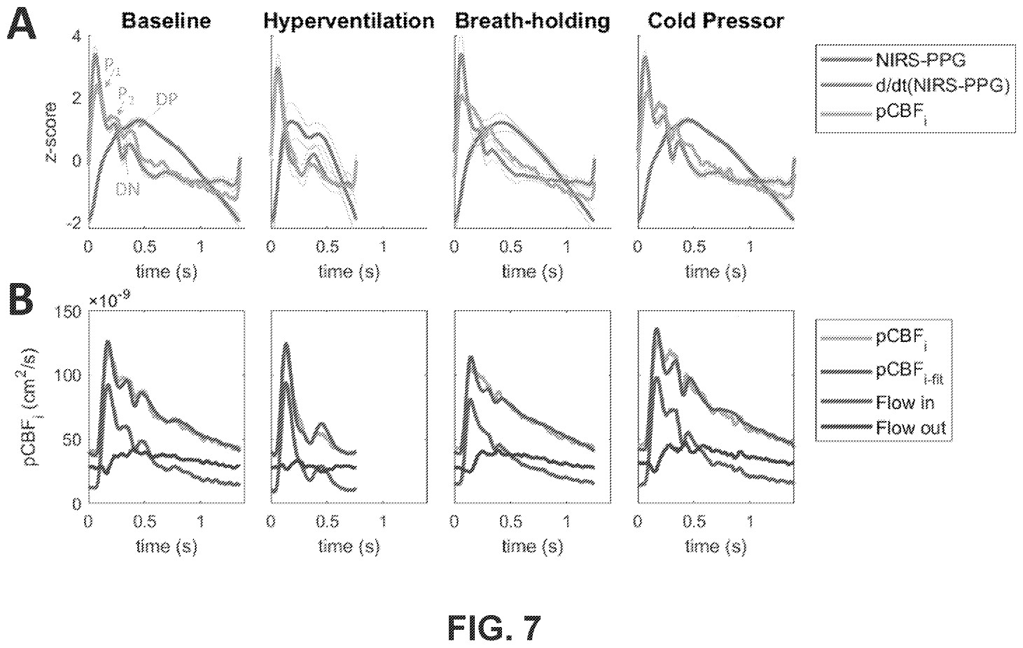

Cardiac-gated averaging of DCS and FlexNIRS signals were performed for each subject and condition. Examples of pCBFi, NIRS-PPG and d (NIRS-PPG)/dt on a representative subject (#5) are shown in FIG. 7A. The 4 panels show the pulsatile waveform during baseline, hyperventilation, breath-holding and cold pressor conditions. We report the pulse waveform in normalized z-scores, which is defined as the number of standard deviations away from the mean (calculated as z=(xi−μ)/σ. xi: data points, μ: mean, σ: standard deviation). We observed a strong similarity between pCBFi and d (NIRS-PPG)/dt, with comparable morphology changes of the main peaks with different conditions. Across all subjects and conditions, the R2 between pCBFi and d (NIRS-PPG)/dt was 0.89±0.10.

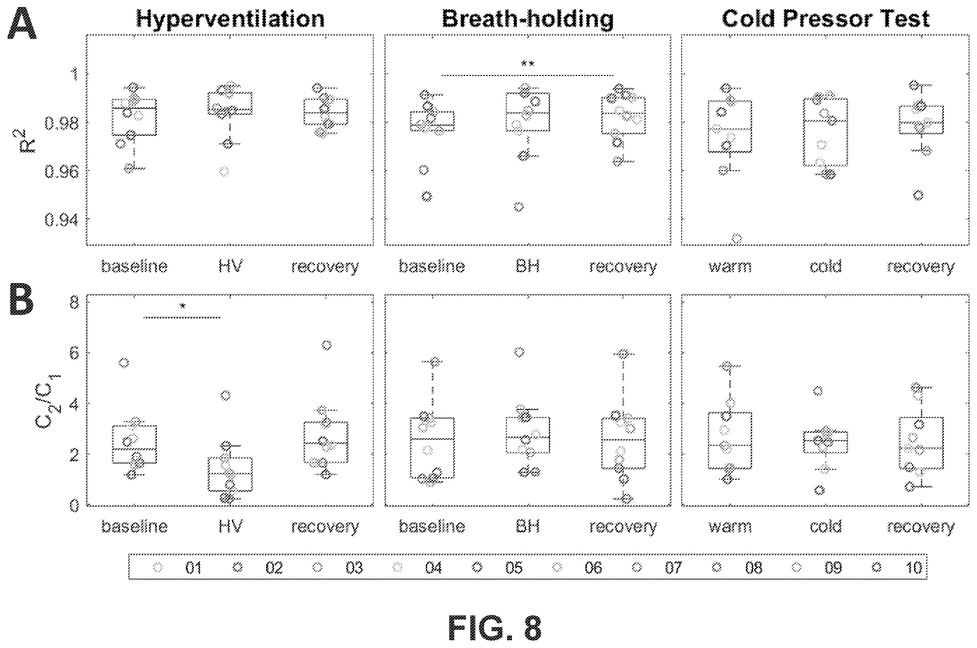

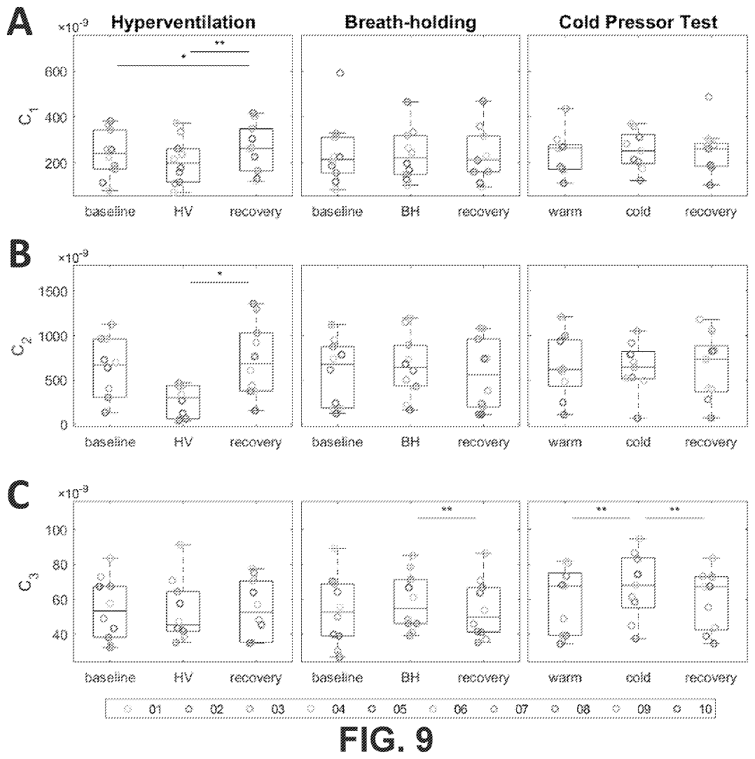

The difference between pCBFi and d (NIRS-PPG)/dt can be accounted for by incorporating the NIRS-PPG waveform contribution. Using Eq. 4-6, we were able to reconstruct pCBFi as pCBFi-fit using NIRS-PPG and separate inflow and outflow (FIG. 7B, results for the same representative subject). We found pCBFi-fit to be nearly identical to pCBFi, with R2 of 0.98±0.01 across all subjects and conditions (FIG. 8A). The fitting parameters C1 and C2 are reported in FIG. 9, and the ratio C2/C1 is shown in FIG. 8B. The parameters C1 and C2 differ between subjects, but weakly depend on different condition, except for hyperventilation, where C1, C2 and C2/C1 dropped (for C2/C1 relative to baseline, p<0.05, paired-sample sign test).

With the higher SNR of the pCBFi-fit, we could identify waveform features automatically. We quantified the systolic peak (P1), secondary systolic peaks (P2, tidal wave), dicrotic notch (DN) and diastolic peak (DP) amplitudes and time delay. As an example, the value of P1, P2, and P2/P1 across subjects and conditions are reported in FIG. 10. We found P2/P1 dropped during hyperventilation (relative to recovery, p<0.01, paired-sample sign test) and increased during cold pressor test (relative to baseline, p<0.01, paired-sample sign test).

### Minimal Cardiac-Gated Averaging Noise Characterization

To quantify the improvement in SNR of pCBFi-fit with respect to SNSPD-DCS pCBFi, we evaluated the coefficient of variation of the pulsatile waveforms during a 60-s baseline. FIG. 8A shows the average and standard deviation of pCBFi and pCBFi-fit for each subject. The shapes of the pulsatile waveforms were diverse among subjects, yet the NIRS-PPG fits showed a strong agreement with the DCS-measured pCBFi. pCBFi-fit provided an improvement on the CV, as the CV of pCBFi-fit was consistently lower than the CV of pCBFi, and on average CVpCBFi/CVpCBFi-fit=3.1±1.7 (FIG. 11). Paired-sample sign test rejected the null hypothesis with p<0.01.

### Discussion

We demonstrated that the simultaneous use of NIRS and DCS allows for the recovery of pulsatile blood flow waveforms with a higher SNR and a higher temporal resolution than with DCS alone, even when using a high-end DCS system like the SNSPD-DCS at 1064 nm here. The use of a low noise, high sampling rate NIRS device such as FlexNIRS (266 Hz) allowed for a 3 times improvement in pCBFi-fit CV with respect to SNSPD DCS pCBFi (FIG. 11) and an increase in sampling rate from 100 to 266 Hz at >3 cm SD separation. Moreover, the low noise of the recovered pCBFi-fit allowed us to use automatic pulsatile features extraction algorithms for PWA. This method was demonstrated with SNSPD DCS in 10 healthy subjects, showing that a 3-parameter linear fitting model using the NIRS-PPG and its time derivative signals is sufficient to recover pCBFi with high accuracy (R2-0.98) (FIG. 7). We showed that the fitting parameters are quite stable within the same subject, despite induced physiological changes (FIG. 8), which should allow for the use of the method with common DCS systems, for which a much larger cardiac gated averaging is needed to provide a pCBFi waveform, especially at 3 cm source-detector separations.

Because of the similarity of pCBFi and d (NIRS-PPG)/dt (R2=0.89), when performing PWA, if necessary, d (NIRS-PPG)/dt alone can be used to approximate pCBFi. Instead, to calculate CrCP, CVRi and PI, the pCBFi mean amplitude is needed to calibrate d (NIRS-PPG)/dt, so a combined NIRS-DCS approach is necessary. The use of pCBFi-fit allows for calculating CrCP, CVRi and PI with minimal averaging and detecting changes during transients. The 4 s average was done to reduce pulse-to-pulse variability and to obtain a more stable fitting when estimating CrCP. In the literature, CVRi is often calculated using the mean CBF and the mean blood pressure values, which requires the assumption of a zero intercept on the pressure-flow relationship. This is not always the case as shown by the high CrCP values (the pressure-intercept) and changes with tasks. It is interesting to see that while the PI is often used as a measure of cerebrovascular resistance, CVRi and PI do not always match during task conditions, likely because the pulsatility index is also affected by the compliance of the cerebral arterial bed, arterial blood pressure and heart rate (53) which differentially vary during the selected tasks.

As shown in FIG. 7, this method allows for the separation of inflow and outflow contributions to the pulsatile flow. The separation of the individual contributions may be useful when trying to estimate intracranial pressure using pulsatile features, since increases in ICP would likely affect the lower-pressure venous outflow more than, and in a different way to, the higher-pressure arterial inflow.

With respect to the slow hemodynamic signal's changes measured during the various tasks, our findings are in agreement with what was expected based on literature reports. Hemoglobin concentration changes during hyperventilation (54,55) and breath-holding (56) are consistent with previous NIRS findings. The CBFi response during hyperventilation and breath-holding is consistent with blood flow results previously reported by our group (17). The decrease in CVRi and PI during breath holding are consistent with findings of transcranial Doppler ultrasound studies (57-59). During the cold pressor tasks, the measured increases in cerebral blood flow, hemoglobin oxygenation and total hemoglobin concentration are due to systemic blood pressure increases above cerebral autoregulation thresholds, and, in the frontal area, are also due to functional activity caused by pain stimulation (60). CBF increases during CPT were previously observed with TCD (61), and cerebral oxygenation increase aligns with prior NIRS studies (62-64). Similarly, during cold pressor tasks, decreases in PI have been observed with TCD (65,66), and the increases we observed in CVRi are consistent with TCD-derived resistance area product (RAP) increases reported by Panerai et al. (67).

This study has several limitations. Because of optical crosstalk, we could not measure NIRS and DCS in the same location, but instead contralaterally across the forehead. This may account for the small differences we observed between the pCBFi and pCBFi-fit waveforms. This problem can be resolved in the future by unifying the NIRS and DCS systems using a single probe and temporal multiplexing, by pulsing the DCS laser source.

The NIRS-PPG fitting procedure is affected by having several local minima, especially for the NIRS-PPG contribution term C2. This is because the NIRS-PPG waveform has fewer distinct features and contribute less than the first derivative. We overcame this problem by sweeping the fitting parameters.

To demonstrate the method, we used a high-end DCS device with a more than 16 times higher SNR than conventional DCS. When using conventional DCS at traditional NIRS wavelengths, the recovered pCBFi will be much noisier, even after averaging several cardiac cycles. This may cause larger errors in determining the C1 and C2 fitting parameters.

Cardiac-gated averaging introduces error, especially when the heartrate varies. Using the combined NIRS-DCS approach we were able to substantially reduce the number of cardiac-gated averaging needed to obtain clean pulsatile blood flow signals.

In conclusion, the use of NIRS-PPG and d (NIRS-PPG)/dt allows to achieve pulsatile blood flow with high temporal resolution and minimal cardiac-gated averaging. Combined with DCS, to obtain calibration, it allows us to derive CrCP and CVRi at a rate of 4 s. The combination of FlexNIRS and SNSPD-DCS at 1064 nm allows for the comprehensive probing of hemodynamic response during breathing and other tasks.

### Example 3: Reiteration of the Non-Limiting Embodiments Described in Example 2: Enhancing Diffuse Correlation Spectroscopy Pulsatile Cerebral Blood Flow Signal with Near-Infrared Spectroscopy Photoplethysmography

The following Example reiterates the non-limiting embodiments described in Example 2.

In certain embodiments, to enhance the signal-to-noise ratio and sampling rate of collected SPG data, a simultaneous collection of PPG data and

$$\frac{d P P G}{dt}$$

data can be used to estimate a more reliable SPG signal using the vascular in-flow and out-flow relationship described in PCT WO2016164894.

The time derivative of the PPG signal is proportional to the volumetric flow rate in the tissue, described as,

$${F}_{in} - {F}_{out} = γ * \frac{d P P G}{dt} .$$

The SPU signal measured by either DCS or SCOS is proportional to the total blood flow in the tissue, described as, Fᵢₙ+Fₒᵤₜ=κ*SPG. Finally, the out-flow component is described as a passive process proportional to the volume of blood in the vasculature, given as Fₒᵤₜ=a*PPG+b. Combining these expressions allows for the description of the SPG signal in terms of the PPG signal and its time derivative,

$$\frac{d P P G}{dt} ,$$

given as

$$S P G = \frac{γ}{κ} \frac{d P P G}{dt} + \frac{2 a}{κ} P P G + \frac{2 b}{κ} ,$$

which we can simplify by substituting three constants for the constants in the expression

$$( {C}_{1} = \frac{γ}{κ} , {C}_{2} = \frac{2 a}{κ} , {C}_{3} = \frac{2 b}{κ} ) .$$

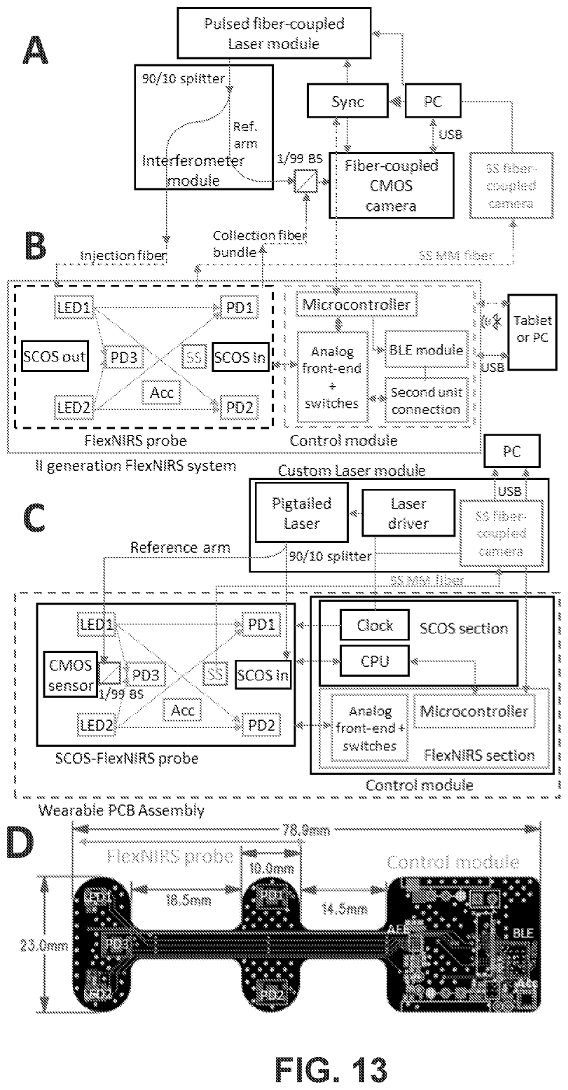

A possible embodiment of a combined measurement system for SPG and PPG is shown in FIG. 6. Another possible embodiment of a combined measurement system for SPG and PPG is shown in FIG. 13.

The DCS system used in this embodiment operates at 1064 nm using a long coherence length laser (CrystaLaser, USA) and four high photon detection efficiency (88%) SNSPD detectors (Opus One, Quantum Opus, USASource and detector fibers were terminated with prisms into a custom optical probe made of flexible 3D-printing material (NinjaTek, USA).

An example of the PPG,

$$\frac{d P P G}{dt} ,$$

and SPG (pCBFᵢ) signals collected during different standards tasks can be seen in FIG. 7A. The combination of the PPG and

$$\frac{d P P G}{dt}$$

are used to fit the SPG data and the overlay across conditions, as well as the decomposition of the flow into in-flow and out-flow components, can be seen in FIG. 7B.

The PPG and

$$\frac{d P P G}{dt}$$

reconstructed SPG waveform is demonstrated to fit very well to the DCS derived SPG waveform (pCBFᵢ) across 10 measured subjects (FIG. 8A), with a stable ratio of C₂ and C₁ across tasks and subjects (FIG. 8B), indicating the relative contributions of PPG and

$$\frac{d P P G}{dt}$$

to the SPG waveform are stable.

The PPG and

$$\frac{d P P G}{dt}$$

reconstructed SPG waveform is demonstrated to have a higher signal-to-noise ratio than the DCS derived SPG waveform (FIGS. 11A and 11B), while also exhibiting a higher sampling rate, allowing for the extraction of more fine-grained features.

The NIRS enhanced DCS measurement of the SPG signal allows for higher SNR measurements of the SPG signal with more temporal detail, improving the rate at which a reliable SPG waveform can be calculated, in this example shortening the averaging time from 15 s to 4 s. and increasing the possible number of features that can be extracted from the SPG waveform in addition to P₂ and P₁.

### Example 4: System for Measuring Blood Pressure Using DCS-SPG and NIRS-PPG

The following Example describes a non-limiting embodiment of the systems disclosed herein.

The DCS system used in this embodiment may operate at 1064 nm using a long coherence length laser (CrystaLaser, USA) and four high photon detection efficiency (88%) SNSPD detectors (Opus One, Quantum Opus, USASource) and detector fibers terminated with prisms into a custom optical probe made of flexible 3D-printing material (NinjaTek, USA).

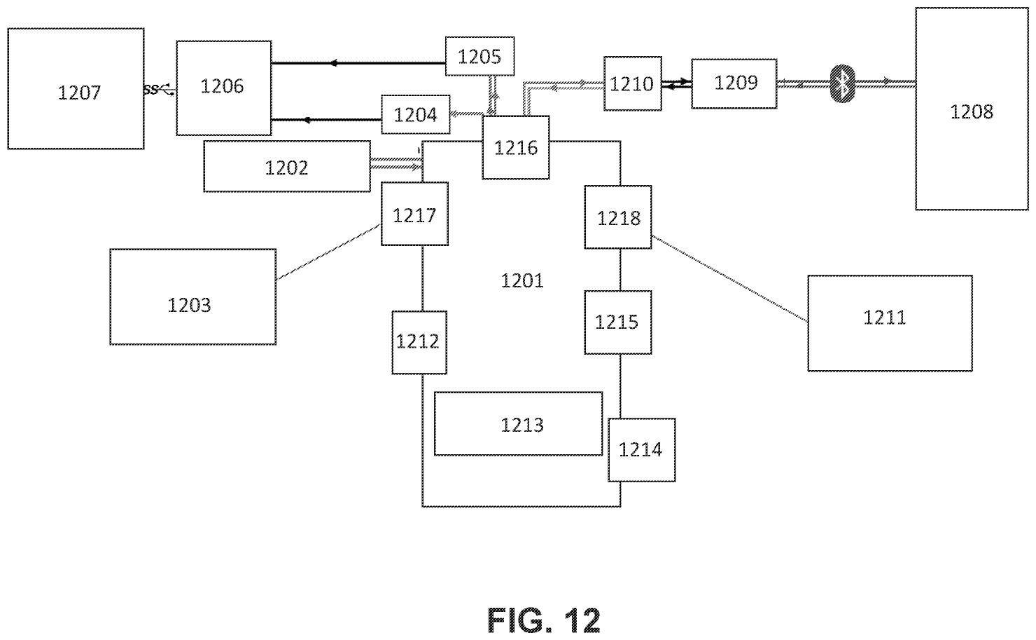

FIG. 12 illustrates a blood pressure measurement setup for a subject (1201) comprising a laser (1202) that transmits a light signal through a DCS-SPG probe (1203). Signals may then transmit divergently through Si SPAD (1204), for example, and SNSPDs (1205), for example. The signals may transmit to a correlator (1206) connected to a computer (1207) by USB, for example. An Android tablet (1208), for example, transmits signal via Bluetooth to a BLE module (1209), for example, and AFE module (1210), for example, NIRS-PPG probe (1211). The signal is transmitted back through the AFE module (1210) and BLE module (1209) back to the Android tablet (1208). Estimation of blood pressure may be conducted through continuous or non-continuous non-invasive volume-clamp methods including Finapres devices, for example (1212), and optional respiration belt (1213), pulse oximeter (1214), and/or ECG (1215) may be used. Sensors may be placed at exemplary locations such as forehead (1216), earlobe (1217), and/or neck (1218).

In certain embodiments, to enhance the signal-to-noise ratio and sampling rate of collected SPG data, a simultaneous collection of PPG data and

$$\frac{d P P G}{dt}$$

data can be used to estimate a more reliable SPG signal using the vascular in-flow and out-flow relationship.

The time derivative of the PPG signal is proportional to the volumetric flow rate in the tissue, described as,

$${F}_{in} - {F}_{out} = γ * \frac{d P P G}{dt} .$$

The SPG signal measured by either DCS or SCOS is proportional to the total blood flow in the tissue, described as, Fᵢₙ+Fₒᵤₜ=κ*SPG. Finally, the out-flow component is described as a passive process proportional to the volume of blood in the vasculature, given as Fₒᵤₜ=a*PPG+b. Combining these expressions allows for the description of the SPG signal in terms of the PPG signal and its time derivative,

$$\frac{d P P G}{dt} ,$$

given as

$$S P G = \frac{γ}{κ} \frac{d P P G}{dt} + \frac{2 a}{κ} P P G + \frac{2 b}{κ} ,$$

which we can simplify by substituting three constants for the constants in the expression

$$( {C}_{1} = \frac{γ}{κ} , {C}_{2} = \frac{2 a}{κ} , {C}_{3} = \frac{2 b}{κ} ) .$$

### Example 5: Estimating Mean Arterial Pressure Using NIRS Car Clip Device

Mean arterial pressure (MAP) can be accurately estimated using

$$\frac{d P P G}{dt}$$

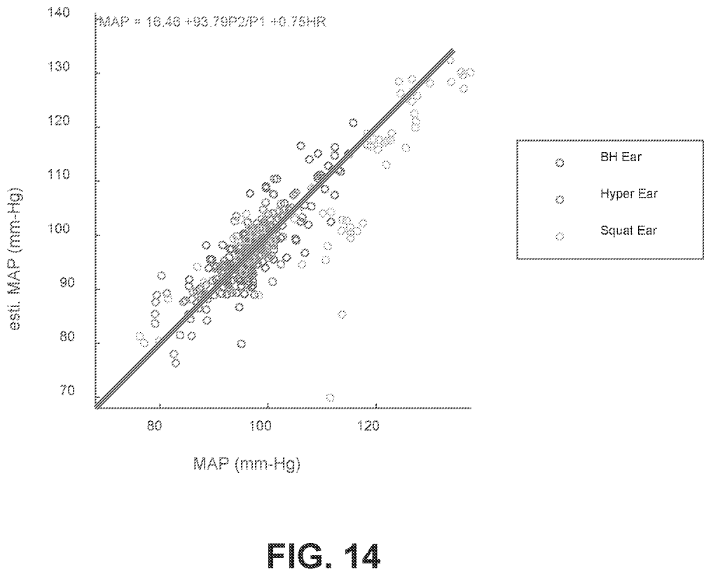

multivariate regression method with P2/P1 and heart rate as inputs. A scatterplot of estimated vs actual mean blood pressure measured in the ear of a subject using an ear clip NIRS device is illustrated in FIG. 14.

### Example 6: Blood Pressure Estimations Using FlexNIRS on the Forehead of a Surgical Patient Correlates with Systolic Blood Pressure

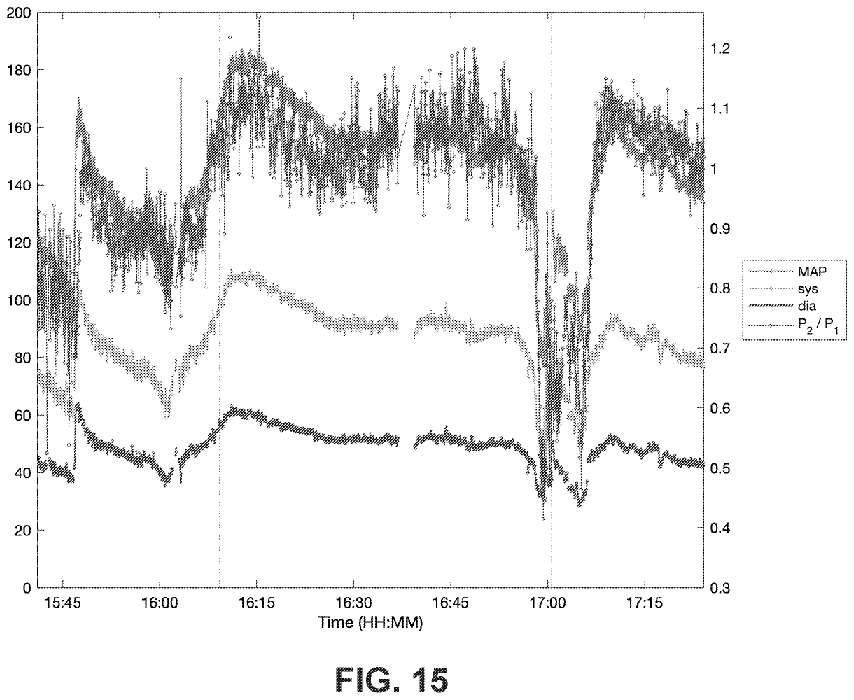

Blood pressure (mean, systolic, and diastolic) was continuously measured with an a-line in a patient during a carotid enterectomy surgery (FIG. 15). The

$$\frac{d P P G}{dt}$$

P2/P1 ratio measured with the FlexNIRS in the forehead of the patient (ipsilateral to the surgery side) estimated a systolic blood pressure that correlated to the a-line-measured blood pressure. SPG was not measured in this patient. Flow in and flow out will further allow to estimate diastolic pressure.

Applicant discloses the following embodiments:

Embodiment 1: An optical patient monitoring system comprising: an optical coupling system configured to transmit and receive light signals at one or more locations on a subject; an optical processing system configured to generate optical data using the received light signals; and a computer programmed to: receive the optical data; determine, using the optical data, at least one indicator of blood pressure; estimate an estimated blood pressure using the at least one indicator of blood pressure; and generate a report indicative of the estimated blood pressure.

Embodiment 2: The system of embodiment 1, wherein the at least one indicator of blood pressure comprises one or more of: near-infrared spectroscopy (NIRS) data; photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative

$$\frac{{d}^{2} P P G}{{dt}^{2}}$$

data, third derivative

$$\frac{{d}^{3} P P G}{{dt}^{3}}$$

data, first derivative

$$\frac{d S P G}{dt}$$

data, second derivative

$$\frac{{d}^{2} S P G}{{dt}^{2}}$$

data, inflow (Fᵢₙ) data, outflow (Fₒᵤₜ) data, heart rate data, physiological data, and combinations thereof.

Embodiment 3: The system of any one of the preceding embodiments, wherein the at least one indicator of blood pressure comprises one or more of: photoplethysmography (PPG) data, first derivative

$$\frac{d P P G}{dt}$$

data, second derivative

$$\frac{{d}^{2} P P G}{{dt}^{2}}$$

data, and third derivative

$$\frac{{d}^{3} P P G}{{dt}^{3}}$$

data, and the optical coupling system comprises a light source comprising at least one of an LED or photodiode in a NIRS device, pulse oximeter or cerebral oximeter.

Embodiment 4: The system of any one of the preceding embodiments, wherein the at least one indicator of blood pressure comprises one or more of: speckleplethysmography (SPG) data, first derivative

$$\frac{d S P G}{dt}$$

data, and second derivative

$$\frac{{d}^{2} S P G}{{dt}^{2}}$$

data, and the optical coupling system comprises a light source comprising a laser.

Embodiment 4: The system of embodiment 2, wherein the physiological data comprises at least one of electrocardiogram (ECG) data, electroencephalogram (EEG) data, near infrared spectroscopy (NIRS) data, measured blood pressure data, respiratory data, hemoglobin data, pulse oximetry data, tissue oxygenation data, heart rate data, or combinations thereof.

Embodiment 6: The system of any one of the preceding embodiments, wherein the at least one indicator of blood pressure comprises at least DCS data and NIRS data.

Embodiment 7: The system of any one of the preceding embodiments, wherein the at least one indicator of blood pressure comprises a ratio of the magnitude of a pre-dicrotic peak (P₂) and a systolic peak (P₁) for at least one individual cardiac cycle in at least one of a PPG curve, a

$$\frac{d P P G}{dt}$$

curve, an SPG curve, or a

$$\frac{d S P G}{dt}$$

curve.

Embodiment 8: The system of any one of the preceding embodiments, wherein the at least one indicator of blood pressure comprises one or more of subject height, subject weight, subject age, subject gender, subject body position data, subject location measured data, and combinations thereof.

Embodiment 9: The system of any one of the preceding embodiments, wherein the optical data is acquired over a plurality of cardiac cycles.

Embodiment 10: The system of any one of the preceding embodiments, wherein the optical data is acquired at a temporal resolution greater than a pulsatile frequency of the subject.

Embodiment 11: The system of any one of the preceding embodiments, wherein the blood pressure is at least one of systolic blood pressure (SBP), diastolic blood pressure (DBP), and mean arterial pressure (MAP).

Embodiment 12: The system of any one of the preceding embodiments, wherein the computer is configured to estimate the estimated blood pressure by performing a calibration against a measured blood pressure data set.

Embodiment 13: The system of embodiment 12, wherein the measured blood pressure data set is acquired by at least one of continuous measurements, non-continuous measurements, invasive (intra-arterial) blood pressure monitoring, non-invasive volume-clamp method, cuff point measurements, automated oscillometry, manual auscultation, and combinations thereof.

Embodiment 14: The system of any one of the preceding embodiments, wherein the computer is configured to apply a machine learning algorithm to estimate the estimated blood pressure by analyzing a measured blood pressure data set against at least one of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative

$$\frac{{d}^{2} P P G}{{dt}^{2}}$$

data, first derivative

$$\frac{d S P G}{dt}$$

data, second derivative

$$\frac{{d}^{2} S P G}{{dt}^{2}}$$

data, inflow (Fᵢₙ) data, outflow (Fₒᵤₜ) data, heart rate data, a ratio of the magnitude of a pre-dicrotic peak (P₂) and a systolic peak (P₁), and combinations thereof.

Embodiment 15: The system of embodiment 14, wherein the machine learning algorithm approach is at least one of random forests, support vector machines, gaussian process regression, and deep belief networks, and combination thereof.

Embodiment 16: The system of any one of the preceding embodiments, wherein the one or more locations on a subject comprise the subject's head, forehead, neck, earlobe, and combinations thereof.

Embodiment 16: The system of embodiment 2, wherein the heart rate data comprises instantaneous heart rate data.

Embodiment 18: The system of any one of the preceding embodiments, wherein the at least one indicator of blood pressure is acquired during a portion of each cardiac cycle.

Embodiment 19: The system of any one of the preceding embodiments, wherein the computer is further configured to determine an effectiveness of an administered treatment using the estimated blood pressure.

Embodiment 20: The system of any one of the preceding embodiments, wherein the computer is further configured to determine a condition of the subject based on the estimated blood pressure.

Embodiment 21: The system of any one of the preceding embodiments, wherein the transmitted light signal is from a light-emitting diode (LED) source, laser, or combinations thereof.

Embodiment 22: The system of any one of the preceding embodiments, wherein the light signals are transmitted from an optical source and received by a photodetector that are placed between 1 mm and 40 mm apart.

Embodiment 23: The system of any one of the preceding embodiments, wherein the light signals have wavelengths between about 300 nm and about 2000 nm.

Embodiment 24: The system of any one of the preceding embodiments, wherein the optical source is coherent.

Embodiment 25: The system of any one of the preceding embodiments, wherein the level of coherence and source output power is determined by the separation of the source and detector.

Embodiment 26: The system of any one of the preceding embodiments, wherein the light signal is detected by a detector selected from the group comprising PIN photodiodes, avalanche photodiodes (APDs), single-photon avalanche diodes (SPADs), photomultiplier tubes (PMTs), silicon photomultipliers (SiPMs), charge-coupled device (CCD), complementary metal-oxide-semiconductor (CMOS) camera sensors, and combinations thereof.

Embodiment 27: The system of any one of the preceding embodiments, wherein the light signal is detected by a detector selected from the group comprising single-photon avalanche diodes (SPADs), avalanche photodiodes (APDs), silicon photomultipliers (SiPMs), super conducting nanowire detectors (SNSPDs), photomultiplier tubes (PMTs), high frame rate photodiodes, camera sensors, and combinations thereof.

Embodiment 28: The system of any one of the preceding embodiments, wherein the at least one indicator of blood pressure comprises SCOS data.

Embodiment 29: The system of any one of the preceding embodiments, wherein the light signal is detected by a detector selected from the group comprising photodiodes arrays, electron multiplying cameras, intensified cameras, standard CCD cameras, CMOS cameras, and combinations thereof.

Embodiment 30: A method for estimating blood pressure, the method comprising:
transmitting and receiving light signals from one or more locations on a subject using an optical coupling system configured to transmit and receive light signals;
determining, using optical data generated using the received light signals using an optical processing system, at least one indicator of blood pressure;
estimating blood pressure of the subject using the at least one indicator of blood pressure.

Embodiment 31: The method of claim 30, further comprising generating a report indicative of the estimated blood pressure.

Embodiment 32: The method of any one of claims 30-31, wherein the at least one indicator of blood pressure comprises one or more of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative

$$\frac{{d}^{2} P P G}{{dt}^{2}}$$

data, first derivative

$$\frac{d S P G}{dt}$$

data, second derivative

$$\frac{{d}^{2} S P G}{{dt}^{2}}$$

data, inflow (Fᵢₙ) data, outflow (Fₒᵤₜ) data, heart rate data, physiological data, and combinations thereof.

Embodiment 33: The method of any one of claims 30-32, wherein the at least one indicator of blood pressure comprises one or more of: photoplethysmography (PPG) data, first derivative

$$\frac{d P P G}{dt}$$

data, second derivative

$$\frac{{d}^{2} P P G}{{dt}^{2}}$$

data, and third derivative

$$\frac{{d}^{3} P P G}{{dt}^{3}}$$

data, and the optical coupling system comprises a light source comprising at least one of an LED or photodiode in a NIRS device, pulse oximeter or cerebral oximeter.

Embodiment 34: The method of any one of claims 30-33, wherein the at least one indicator of blood pressure comprises one or more of: speckleplethysmography (SPG) data, first derivative

$$\frac{d S P G}{dt}$$

data, and second derivative

$$\frac{{d}^{2} S P G}{{dt}^{2}}$$

data, and the optical coupling system comprises a light source comprising a laser.

Embodiment 35: The method of any one of claims 30-34, wherein the physiological data comprises at least one of electrocardiogram (ECG) data, electroencephalogram (EEG) data, near infrared spectroscopy (NIRS) data, measured blood pressure data, respiratory data, hemoglobin data, pulse oximetry data, tissue oxygenation data, heart rate data, or a combination thereof.

Embodiment 36: The method of any one of claims 30-35, wherein the at least one indicator of blood pressure comprises at least DCS data and NIRS data.

Embodiment 37: The method of any one of claims 30-36, wherein the at least one indicator of blood pressure comprises a ratio of the magnitude of a pre-dicrotic peak (P₂) and a systolic peak (P₁) for at least one individual cardiac cycle in at least one of a Pro curve, a

$$\frac{d P P G}{dt}$$

curve, an SPG curve, or a

$$\frac{d S P G}{dt}$$

curve.

Embodiment 38: The method of any one of claims 30-37, wherein the at least one indicator of blood pressure comprises one or more of subject height, subject weight, subject age, subject gender, subject body position data, subject location measured data, and combinations thereof.

Embodiment 39: The method of any one of claims 30-38, wherein the optical data is acquired over a plurality of cardiac cycles.

Embodiment 40: The method of any one of claims 30-39, wherein the optical data is acquired at a temporal resolution greater than a pulsatile frequency of the subject.

Embodiment 41: The method of any one of claims 30-40, wherein the blood pressure is at least one of systolic blood pressure (SBP), diastolic blood pressure (DBP), and mean arterial pressure (MAP).

Embodiment 42: The method of any one of claims 30-41, further comprising performing a calibration against a measured blood pressure data set.

Embodiment 43: The method of any one of claims 30-42, wherein the measured blood pressure data set is acquired by at least one of continuous measurements, non-continuous measurements, invasive (intra-arterial) blood pressure monitoring, non-invasive volume-clamp method, cuff point measurements, automated oscillometry, manual auscultation, and combinations thereof.

Embodiment 44: The method of any one of claims 30-43, further comprising applying a machine learning algorithm to estimate the estimated blood pressure by analyzing a measured blood pressure data set against at least one of photoplethysmography (PPG) data, diffuse correlation spectroscopy (DCS) data, speckle contrast optical spectroscopy (SCOS) data, speckleplethysmography (SPG) data, first derivative

$$\frac{d P P G}{dt}$$

data, second derivative

$$\frac{{d}^{2} P P G}{{dt}^{2}}$$

data, first derivative

$$\frac{d S P G}{dt}$$

data, second derivative

$$\frac{{d}^{2} S P G}{{dt}^{2}}$$

data, inflow (Fᵢₙ) data, outflow (Fₒᵤₜ) data, heart rate data, a ratio of the magnitude of a pre-dicrotic peak (P₂) and a systolic peak (P₁), and combinations thereof.

Embodiment 45: The method of any one of claims 30-44, wherein the machine learning algorithm approach is at least one of random forests, support vector machines, gaussian process regression, and deep belief networks, and combinations thereof.

Embodiment 46: The method of any one of claims 30-45, wherein the one or more locations on a subject comprise the subject's head, forehead, neck, earlobe, and combinations thereof.

Embodiment 47: The method of any one of claims 30-46, wherein the heart rate data comprises instantaneous heart rate data.

Embodiment 48: The method of any one of claims 30-47, wherein the at least one indicator of blood pressure is acquired during a portion of each cardiac cycle.

Embodiment 49: The method of any one of claims 30-48, further comprising determining an effectiveness of an administered treatment using the estimated blood pressure.

Embodiment 50: The method of any one of claims 30-49, further comprising determining a condition of the subject based on the estimated blood pressure.

Embodiment 51: The method of any one of claims 30-50, wherein the transmitted light signal is from a light-emitting diode (LED) source, laser, or combinations thereof.

Embodiment 52: The method of any one of claims 30-51, wherein the light signals are transmitted from an optical source and received by a photodetector that are placed between 1 mm and 40 mm apart.

Embodiment 53: The method of any one of claims 30-52, wherein the light signals have wavelengths between about 300 nm and about 2000 nm. is coherent.

Embodiment 55: The method of any one of claims 30-54, wherein the level of coherence and source output power is determined by the separation of the source and detector.

Embodiment 54: The method of any one of claims 30-53, wherein the optical source

Embodiment 56: The method of any one of claims 30-55, wherein the light signal is detected by a detector selected from the group comprising single-photon avalanche diodes (SPADs), avalanche photodiodes (APDs), silicon photomultipliers (SiPMs), super conducting nanowire detectors (SNSPDs), photomultiplier tubes (PMTs), high frame rate photodiodes, camera sensors, and combinations thereof.

Embodiment 57: The method of any one of claims 30-56, wherein the light signal is detected by a detector selected from the group comprising single-photon avalanche diodes (SPADs), avalanche photodiodes (APDs), silicon photomultipliers (SiPMs), super conducting nanowire detectors (SNSPDs), photomultiplier tubes (PMTs), high frame rate photodiodes, camera sensors, and combinations thereof.

Embodiment 58: The method of any one of claims 30-57, wherein the at least one indicator of blood pressure comprises SCOS data.

Embodiment 59: The method of any one of claims 30-58, wherein the light signal is detected by a detector selected from the group comprising photodiodes arrays, electron multiplying cameras, intensified cameras, standard CCD cameras, CMOS cameras, and combinations thereof.

### REFERENCES

1. Wray S, Cope M, Delpy D T, et al. Characterization of the near infrared absorption spectra of cytochrome aa3 and haemoglobin for the non-invasive monitoring of cerebral oxygenation. Biochim Biophys Acta (BBA)-Bioenergetics 1988; 933:184-192.
2. Jöbsis F F. Noninvasive, infrared monitoring of cerebral and myocardial oxygen sufficiency and circulatory parameters. Science (80-) 1977; 198:1264-1267.
3. Boas D A, Yodh A G. Spatially varying dynamical properties of turbid media probed with diffusing temporal light correlation. JOSA A 1997; 14:192-215.
4. Buckley E M, Parthasarathy A B, Grant P E, et al. Diffuse correlation spectroscopy for measurement of cerebral blood flow: future prospects. Neurophotonics 2014; 1:11009.
5. Mesquita R C, Durduran T, Yu G, et al. Direct measurement of tissue blood flow and metabolism with diffuse optics. Phil Trans R Soc A 2011; 369:4390-4406.
6. Boas D A, Franceschini M A. Haemoglobin oxygen saturation as a biomarker: the problem and a solution. Philos Trans R Soc A Math Phys Eng Sci 2011; 369:4407-4424.
7. Durduran T, Yu G, Burnett M G, et al. Diffuse optical measurement of blood flow, blood oxygenation, and metabolism in a human brain during sensorimotor cortex activation. Opt Lett 2004; 29:1766-1768.
8. Roche-Labarbe N, Fenoglio A, Radhakrishnan H, et al. Somatosensory evoked changes in cerebral oxygen consumption measured non-invasively in premature neonates. Neuroimage 2014; 85:279-286.
9. Dehaes M, Aggarwal A, Lin P-Y, et al. Cerebral oxygen metabolism in neonatal hypoxic ischemic encephalopathy during and after therapeutic hypothermia. J Cereb Blood Flow Metab 2014; 34:87-94.
10. Zavriyev A I, Kaya K, Farzam P, et al. The role of diffuse correlation spectroscopy and frequency-domain near-infrared spectroscopy in monitoring cerebral hemodynamics during hypothermic circulatory arrests. JTCVS Tech 2021; 7:161-177.
11. Wu K-C, Sunwoo J, Sheriff F, et al. Validation of diffuse correlation spectroscopy measures of critical closing pressure against transcranial Doppler ultrasound in stroke patients. J Biomed Opt 2021; 26:36008.
12. Baker W B, Parthasarathy A B, Gannon K P, et al. Noninvasive optical monitoring of critical closing pressure and arteriole compliance in human subjects. J Cereb Blood Flow Metab 2017; 37:2691-2705.
13. Ruesch A, Yang J, Schmitt S, et al. Estimating intracranial pressure using pulsatile cerebral blood flow measured with diffuse correlation spectroscopy. Biomed Opt Express 2020; 11:1462-1476.
14. Relander F A J, Ruesch A, Yang J, et al. Using near-infrared spectroscopy and a random forest regressor to estimate intracranial pressure. Neurophotonics 2022; 9:45001.
15. Fischer J B, Ghouse A, Tagliabue S, et al. Non-invasive estimation of intracranial pressure by diffuse optics: a proof-of-concept study. J Neurotrauma 2020; 37:2569-2579.
16. Lafontant A, Gabrielli E M, Bergonzi K, et al. Comparison of optical measurements of critical closing pressure acquired before and during induced ventricular arrhythmia in adults. Neurophotonics 2022; 9:35004.
17. Ozana N, Zavriyev A I, Mazumder D, et al. Superconducting nanowire single-photon sensing of cerebral blood flow. Neurophotonics 2021; 8:1-14.
18. Early C B, Dewey R C, Pieper H P, et al. Dynamic pressure-flow relationships of brain blood flow in the monkey. J Neurosurg 1974; 41:590-596.
19. Selb J, Boas D A, Chan S-T, et al. Sensitivity of near-infrared spectroscopy and diffuse correlation spectroscopy to brain hemodynamics: simulations and experimental findings during hypercapnia. Neurophotonics 2014; 1:015005.
20. Zhou C, Yu G, Furuya D, et al. Diffuse optical correlation tomography of cerebral blood flow during cortical spreading depression in rat brain. Opt Express 2006; 14:1125.
21. Strangman G E, Zhang Q, Marshall-Goebel K, et al. Increased cerebral blood volume pulsatility during head-down tilt with elevated carbon dioxide: the SPACECOT Study. J Appl Physiol 2017; 123:62-70.
22. Wu K-C, Tamborini D, Renna M, et al. Open-source FlexNIRS: A low-cost, wireless and wearable cerebral health tracker. Neuroimage 2022; 256:119216.
23. Kyriacou P A, Allen J. Photoplethysmography: Technology, Signal Analysis and Applications. Academic Press, 2021.
24. Millasseau S C, Kelly R P, Ritter J M, et al. Determination of age-related increases in large artery stiffness by digital pulse contour analysis. Clin Sci 2002; 103:371-377.
25. Allen J, Murray A. Age-related changes in the characteristics of the photoplethysmographic pulse shape at various body sites. Physiol Meas 2003; 24:297.
26. Yousef Q, Reaz MBI, Ali MAM. The analysis of PPG morphology: investigating the effects of aging on arterial compliance. Meas Sci Rev 2012; 12:266.
27. Park J, Seok H S, Kim S-S, et al. Photoplethysmogram Analysis and Applications: An Integrative Review. Front Physiol 2022; 2511.
28. Fabiani M, Low K A, Tan C, et al. Taking the pulse of aging: Mapping pulse pressure and elasticity in cerebral arteries with optical methods. Psychophysiology 2014; 51:1072-1088.
29. Chiarelli A M, Fletcher M A, Tan C H, et al. Individual differences in regional cortical volumes across the life span are associated with regional optical measures of arterial elasticity. Neuroimage 2017; 162:199-213.
30. Mohammadi H, Gagnon C, Vincent T, et al. Longitudinal impact of physical activity on brain pulsatility index and cognition in older adults with cardiovascular risk factors: a NIRS study. Brain Sci 2021; 11:730.
31. Cook L B. Extracting arterial flow waveforms from pulse oximeter waveforms: apparatus. Anaesthesia 2001; 56:551-555.
32. Wisely N A, Cook L B. Arterial flow waveforms from pulse oximetry compared with measured Doppler flow waveforms: apparatus. Anaesthesia 2001; 56:556-561.
33. Themelis G, D′Arceuil H E, Diamond S G, et al. Near-infrared spectroscopy measurement of the pulsatile component of cerebral blood flow and volume from arterial oscillations. J Biomed Opt 2007; 12:14033.
34. Siegert A J F. On the fluctuations in signals returned by many independently moving scatterers. Radiation Laboratory, Massachusetts Institute of Technology, 1943.
35. Hines E A. A Clinical Test of Vasomotor Irritablilty: Blood Pressure Response to Cold.
36. Hueber D M, Fantini S, Cerussi A E, et al. New optical probe designs for absolute (self-calibrating) NIR tissue hemoglobin measurements. In: Chance B, Alfano R R, Tromberg B J (eds) Optical Tomography and Spectroscopy of Tissue III. SPIE, 1999, pp. 618-631.
37. Blaney G, Donaldson R, Mushtak S, et al. Broadband absorption spectroscopy in tissue: combining dual-slope continuous-wave and self-calibrating frequency-domain measurements. In: Optical Tomography and Spectroscopy of Tissue XIV. SPIE, 2021, pp. 68-74.
38. Hallacoglu B, Sassaroli A, Fantini S, et al. Absolute measurement of cerebral optical coefficients, hemoglobin concentration and oxygen saturation in old and young adults with near-infrared spectroscopy. J Biomed Opt 2012; 17:81406.
39. Fatouros P P, Marmarou A. Use of magnetic resonance imaging for in vivo measurements of water content in human brain: method and normal values. J Neurosurg 1999; 90:109-115.
40. Boas D A, Campbell L E, Yodh A G. Scattering and imaging with diffusing temporal field correlations. Phys Rev Lett 1995; 75:1855.
41. Carp S A, Tamborini D, Mazumder D, et al. Diffuse correlation spectroscopy measurements of blood flow using 1064 nm light. J Biomed Opt 2020; 25:97003.
42. Delpy D T, Cope M, Van Der Zee P, et al. Estimation of optical pathlength through tissue from direct time of flight measurement. Phys Med Biol 1988; 33:1433-1442.
43. De Riva N, Budohoski K P, Smielewski P, et al. Transcranial doppler pulsatility index: What it is and what it isn't. Neurocrit Care 2012; 17:58-66.
44. Bellner J, Romner B, Reinstrup P, et al. Transcranial Doppler sonography pulsatility index (P I) reflects intracranial pressure (ICP). Surg Neurol 2004; 62:45-51.
45. Raichle M E, Plum F. Hyperventilation and cerebral blood flow. Stroke 1972; 3:566-575.
46. Gouvea Bogossian E, Peluso L, Creteur J, et al. Hyperventilation in adult TBI patients: how to approach it? Front Neurol 2021; 11:580859.
47. Wilson D F, Pastuszko A, DiGiacomo J E, et al. Effect of hyperventilation on oxygenation of the brain cortex of newborn piglets. J Appl Physiol 1991; 70:2691-2696.
48. Parkes M J, Green S, Stevens A M, et al. Assessing and ensuring patient safety during breath-holding for radiotherapy. Br J Radiol 2014; 87:20140454.
49. Perini R, Tironi A, Gheza A, et al. Heart rate and blood pressure time courses during prolonged dry apnoea in breath-hold divers. Eur J Appl Physiol 2008; 104:1-7.
50. Zerweck L, Hauser T-K, Roder C, et al. Investigation of the BOLD-based MRI signal time course during short breath-hold periods for estimation of the cerebrovascular reactivity. S N Compr Clin Med 2020; 2:1551-1562.

51 Larsen J, Kallenberg K, Rühlmann J, et al. Breath holding for 20 s following extended expiration is a practical, effective and robust standard when measuring cerebrovascular reactivity in healthy adults using BOLD fMRI at 3 T. Neuroimage: Reports 2021; 1:100021.
52. Davies H J, Williams I, Peters N S, et al. In-ear spo2: A tool for wearable, unobtrusive monitoring of core blood oxygen saturation. Sensors 2020; 20:4879.
53. Varsos G V, Kolias A G, Smielewski P, et al. using critical closing pressure. 2015; 123:638-648.
54. Sandru S, Buzescu D, Zahiu CDM, et al. Near-Infrared Spectroscopy Usefulness in Validation of Hyperventilation Test. Medicina (B Aires) 2022; 58:1396.
55. Matsuo K, Watanabe A, Onodera Y, et al. Prefrontal hemodynamic response to verbal-fluency task and hyperventilation in bipolar disorder measured by multi-channel near-infrared spectroscopy. J Affect Disord 2004; 82:85-92.
56. Raitamaa L, Korhonen V, Huotari N, et al. Breath hold effect on cardiovascular brain pulsations-A multimodal magnetic resonance encephalography study. J Cereb Blood Flow Metab 2019; 39:2471-2485.
57. van Veen T R, Panerai R B, Haeri S, et al. Effect of breath holding on cerebrovascular hemodynamics in normal pregnancy and preeclampsia. J Appl Physiol 2015; 118:858-862.
58. Settakis G, Lengyel A, Molnár C, et al. Transcranial Doppler study of the cerebral hemodynamic changes during breath-holding and hyperventilation tests. J Neuroimaging 2002; 12:252-258.
59. Hsu H-Y, Chern C-M, Kuo J-S, et al. Correlations among critical closing pressure, pulsatility index and cerebrovascular resistance. Ultrasound Med Biol 2004; 30:1329-1335.
60. La Cesa S, Tinelli E, Toschi N, et al. fMRI pain activation in the periaqueductal gray in healthy volunteers during the cold pressor test. Magn Reson Imaging 2014; 32:236-240.
61. Flück D, Ainslie P N, Bain A R, et al. Extra- and intracranial blood flow regulation during the cold pressor test: influence of age. J Appl Physiol 2017; 123:1071-1080.
62. Barati Z, Shewokis P A, Izzetoglu M, et al. Hemodynamic response to repeated noxious cold pressor tests measured by functional near infrared spectroscopy on forehead. Ann Biomed Eng 2013; 41:223-237.
63. Høiseth L Ø, Hisdal J, Hoff I E, et al. Tissue oxygen saturation and finger perfusion index in central hypovolemia: influence of pain. Crit Care Med 2015; 43:747-756.
64. Barati Z, Zakeri I, Pourrezaei K. Functional near-infrared spectroscopy study on tonic pain activation by cold pressor test. Neurophotonics 2017; 4:15004.
65. Tsai S-J, Chen S-C, Leu T-M, et al. Impairment of cerebral hemodynamic response to the cold pressor test in patients with Parkinson's disease. Parkinsonism Relat Disord 2009; 15:94-100.
66. Micieli G, Tassorelli C, Bosone D, et al. Increased cerebral blood flow velocity induced by cold pressor test in migraine: a possible basis for pathogenesis? Cephalalgia 1995; 15:494-498.
67. Panerai R B, Dawson S L, Eames P J, et al. Cerebral blood flow velocity response to induced and spontaneous sudden changes in arterial blood pressure. Am J Physiol Circ Physiol 2001; 280: H2162-H2174.

We claim:

1. An optical patient monitoring system comprising:
an optical coupling system configured to transmit and receive light signals at one or more locations on a subject;
an optical processing system configured to generate optical data using the received light signals; and
a computer programmed to:
receive the optical data;
determine, using the optical data, at least one indicator of blood pressure;
estimate an estimated blood pressure using the at least one indicator of blood pressure; and
generate a report indicative of the estimated blood pressure.

2. The system of claim 1, wherein the at least one indicator of blood pressure comprises one or more of: near-infrared spectroscopy (NIRS) data; photoplethysmography (PPG) data, diffuse correlation spectroscopy (DCS) data, speckle contrast optical spectroscopy (SCOS) data, speckleplethysmography (SPG) data, first derivative
$$\frac{d P P G}{dt}$$
data, second derivative
$$\frac{{d}^{2} PPG}{{dt}^{2}}$$
data, third derivative
$$\frac{{d}^{3} PPG}{{dt}^{3}}$$
data, first derivative
$$\frac{dSPG}{dt}$$
data, second derivative
$$\frac{{d}^{2} SPG}{{dt}^{2}}$$
data, inflow (Fᵢₙ) data, outflow (Fₒᵤₜ) data, heart rate data, physiological data, and combinations thereof.

3. The system of claim 2, wherein the at least one indicator of blood pressure comprises one or more of: photoplethysmography (PPG) data, first derivative
$$\frac{dPPG}{dt}$$
data, second derivative
$$\frac{{d}^{2} PPG}{{dt}^{2}}$$
data, and third derivative
$$\frac{{d}^{3} PPG}{{dt}^{3}}$$
data, and the optical coupling system comprises a light source comprising at least one of an LED or photodiode in a NIRS device, pulse oximeter or cerebral oximeter.

4. The system of claim 2, wherein the at least one indicator of blood pressure comprises one or more of: speckleplethysmography (SPG) data, first derivative
$$\frac{dSPG}{dt}$$
data, and second derivative
$$\frac{{d}^{2} SPG}{{dt}^{2}}$$
data, and the optical coupling system comprises a light source comprising a laser.

5. The system of claim 2, wherein the physiological data comprises at least one of electrocardiogram (ECG) data, electroencephalogram (EEG) data, near infrared spectroscopy (NIRS) data, measured blood pressure data, respiratory data, hemoglobin data, pulse oximetry data, tissue oxygenation data, heart rate data, or combinations thereof.

6. The system of claim 1, wherein the at least one indicator of blood pressure comprises at least DCS data and NIRS data.

7. The system of claim 1, wherein the at least one indicator of blood pressure comprises a ratio of the magnitude of a pre-dicrotic peak (P₂) and a systolic peak (P₁) for at least one individual cardiac cycle in at least one of a PPG curve, a
$$\frac{dPPG}{dt}$$
curve, an SPG curve, or a
$$\frac{dSPG}{dt}$$
curve.

8. The system of claim 2, wherein the at least one indicator of blood pressure comprises one or more of subject height, subject weight, subject age, subject gender, subject body position data, subject location measured data, and combinations thereof.

9. The system of claim 1, wherein the optical data is acquired over a plurality of cardiac cycles.

10. The system of claim 1, wherein the optical data is acquired at a temporal resolution greater than a pulsatile frequency of the subject.

11. (canceled)

12. The system of claim 1, wherein the computer is configured to estimate the estimated blood pressure by performing a calibration against a measured blood pressure data set.

13. The system of claim 12, wherein the measured blood pressure data set is acquired by at least one of continuous measurements, non-continuous measurements, invasive (intra-arterial) blood pressure monitoring, non-invasive volume-clamp method, cuff point measurements, automated oscillometry, manual auscultation, and combinations thereof.

14. The system of claim 1, wherein the computer is configured to apply a machine learning algorithm to estimate the estimated blood pressure by analyzing a measured blood pressure data set against at least one of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative
$$\frac{dPPG}{dt}$$
data, second derivative
$$\frac{{d}^{2} PPG}{{dt}^{2}}$$
data, first derivative
$$\frac{dSPG}{dt}$$
data, second derivative
$$\frac{{d}^{2} SPG}{{dt}^{2}}$$
data, inflow (Fᵢₙ) data, outflow (Fₒᵤₜ) data, heart rate data, a ratio of the magnitude of a pre-dicrotic peak (P₂) and a systolic peak (P₁), and combinations thereof.

15. The system of claim 14, wherein the machine learning algorithm approach is at least one of random forests, support vector machines, gaussian process regression, and deep belief networks, and combination thereof.

16. The system of claim 1, wherein the one or more locations on a subject comprise the subject's head, forehead, neck, earlobe, and combinations thereof.

17. The system of claim 2, wherein the heart rate data comprises instantaneous heart rate data.

18. The system of claim 1, wherein the at least one indicator of blood pressure is acquired during a portion of each cardiac cycle.

19-21. (canceled)

22. The system of claim 3, wherein the light signals are transmitted from an optical source and received by a photodetector that are placed between 1 mm and 40 mm apart.

23. The system of claim 3, wherein the light signals have wavelengths between about 300 nm and about 2000 nm.

24. The system of claim 4, wherein the optical source is coherent.

25. The system of claim 24, wherein the level of coherence and source output power is determined by the separation of the source and detector.

26. The system of claim 3, wherein the light signal is detected by a detector selected from the group comprising PIN photodiodes, avalanche photodiodes (APDs), single-photon avalanche diodes (SPADs), photomultiplier tubes (PMTs), silicon photomultipliers (SiPMs), charge-coupled device (CCD), complementary metal-oxide-semiconductor (CMOS) camera sensors, and combinations thereof.

27. The system of claim 4, wherein the light signal is detected by a detector selected from the group comprising single-photon avalanche diodes (SPADs), avalanche photodiodes (APDs), silicon photomultipliers (SiPMs), super conducting nanowire detectors (SNSPDs), photomultiplier tubes (PMTs), high frame rate photodiodes, camera sensors, and combinations thereof.

28. The system of claim 2, wherein the at least one indicator of blood pressure comprises SCOS data.

29. (canceled)

30. A method for estimating blood pressure, the method comprising:
transmitting and receiving light signals from one or more locations on a subject using an optical coupling system configured to transmit and receive light signals;
determining, using optical data generated using the received light signals using an optical processing system, at least one indicator of blood pressure; and
estimating blood pressure of the subject using the at least one indicator of blood pressure.

31. (canceled)

32. The method of claim 30, wherein the at least one indicator of blood pressure comprises one or more of: near-infrared spectroscopy (NIRS) data, photoplethysmography (PPG) data, diffuse correlation spectroscopy (DCS) data, speckle contrast optical spectroscopy (SCOS) data, speckleplethysmography (SPG) data, first derivative
$$\frac{d P P G}{dt}$$
data, second derivative
$$\frac{{d}^{2} P P G}{{dt}^{2}}$$
data, first derivative
$$\frac{d S P G}{dt}$$
data, second derivative
$$\frac{{d}^{2} S P G}{{dt}^{2}}$$
data, inflow (Fᵢₙ) data, outflow (Fₒᵤₜ) data, heart rate data, physiological data, and combinations thereof.

33. The method of claim 30, wherein the at least one indicator of blood pressure comprises one or more of: photoplethysmography (PPG) data, first derivative
$$\frac{d P P G}{dt}$$
data, second derivative
$$\frac{{d}^{2} P P G}{{dt}^{2}}$$
data, and third derivative
$$\frac{{d}^{3} P P G}{{dt}^{3}}$$
data, and the optical coupling system comprises a light source comprising at least one of an LED or photodiode in a NIRS device, pulse oximeter or cerebral oximeter.

34. The method of claim 30, wherein the at least one indicator of blood pressure comprises one or more of: speckleplethysmography (SPG) data, first derivative
$$\frac{d S P G}{dt}$$
data, and second derivative
$$\frac{{d}^{2} S P G}{{dt}^{2}}$$
data, and the optical coupling system comprises a light source comprising a laser.

35-36. (canceled)

37. The method of claim 30, wherein the at least one indicator of blood pressure comprises a ratio of the magnitude of a pre-dicrotic peak (P₂) and a systolic peak (P₁) for at least one individual cardiac cycle in at least one of a PPG curve, a
$$\frac{d P P G}{dt}$$
curve, an SPG curve, or a
$$\frac{d S P G}{dt}$$
curve.

38-39. (canceled)

40. The method of claim 30, wherein the optical data is acquired at a temporal resolution greater than a pulsatile frequency of the subject.

41. (canceled)

42. The method of claim 30, further comprising performing a calibration against a measured blood pressure data set.

43. (canceled)

44. The method of claim 30, further comprising applying a machine learning algorithm to estimate the estimated blood pressure by analyzing a measured blood pressure data set against at least one of photoplethysmography (PPG) data, diffuse correlation spectroscopy (DCS) data, speckle contrast optical spectroscopy (SCOS) data, speckleplethysmography (SPG) data, first derivative
$$\frac{d P P G}{dt}$$
data, second derivative
$$\frac{{d}^{2} P P G}{{dt}^{2}}$$
data, first derivative
$$\frac{d S P G}{dt}$$
data, second derivative
$$\frac{{d}^{2} S P G}{{dt}^{2}}$$
data, inflow (Fᵢₙ) data, outflow (Fₒᵤₜ) data, heart rate data, a ratio of the magnitude of a pre-dicrotic peak (P₂) and a systolic peak (P₁), and combinations thereof.

45-57. (canceled)

58. The method of claim 32, wherein the at least one indicator of blood pressure comprises SCOS data.

59. (canceled)
